(19) United States
(12) Patent Application Publication
(10) Pub. No.: US 2005/0124664 A1
(43) Pub. Date: Jun. 9, 2005

(54) Urea thiadiazole inhibitors of plasminogen activator inhibior-1

(76) Inventors: Eric Sartori, Paris (FR); Magali Maillet, Suresne (FR); Marie France Paugam, Fontenay le Fleury (FR); Eric Nicolai, Rueil-Malmaison (FR); Michael Lawrence, Yardley, PA (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/969,703

(22) Filed: Oct. 20, 2004

Related U.S. Application Data
(60) Provisional application No. 60/515,906, filed on Oct. 30, 2003.

(51) Int. Cl. A61K 31/4439; A61K 31/433; C07D 417/02

(52) U.S. Cl. 514/341; 514/362; 546/268.7; 548/128

(57) ABSTRACT

Methods of treating disorders associated with elevated levels of PAI-1 are disclosed comprising administering to a patient in need thereof a therapeutically effective amount of at least one compound of formula (I), 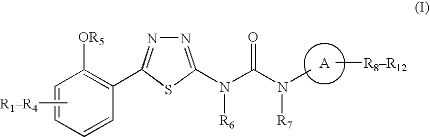
or a pharmaceutically-acceptable salt, prodrug, stereoisomer or solvate thereof, wherein: A is aryl o heteroaryl, and R₁-R₁₂, are defined herein. The invention also pertains to pharmaceutical compositions and compounds within the scope of formula (I) as well as medicaments and articles of manufacture comprising compounds of formula (I).

This applications claims the benefit of priority from U.S. Provisional Application Ser. No. 60/515,906, filed Oct. 30, 2003.

### FIELD OF THE INVENTION

This invention relates to methods of using urea thiadiazole compounds to inhibit Plasminogen Activator Inhibitor-1 (PAI-1 inhibitor) to treat disorders associated with elevated levels of PAI-1, as well as pharmaceutical compositions and articles of manufacture comprising the urea thiadiazole compounds.

### BACKGROUND OF THE INVENTION

Plasminogen activator inhibitor-1 (PAI-1) is a member of the serine protease inhibitor (“SERPIN”) superfamily of proteins and plays a major role in the regulation of the plasminogen-plasmin system. PAI-1 is known to be the principal inhibitor of the serine proteases tissue-type plasminogen activator (t-PA) and urokinase-type plasminogen activator (u-PA). In the plasma, t-PA cleaves the zymogen plasminogen to the active enzyme plasmin, which can degrade fibrin clots (fibrinolysis or thrombolysis) thereby exerting an antithrombotic effect. Therefore, through the regulation of t-PA, PAI-1 plays an important role in hemostasis. An increased level of PAI-1 is believed to be a risk factor in thrombotic conditions such as venous thrombosis, atherosclerosis, and arterial thrombosis, which can result in deep vein thrombosis, pulmonary embolism, myocardial infarction, stroke, etc. See e.g., Wu, Current Drug Targets, 2, 27, (2002); Dawson, et al., Atherosclerosis, 95, 105 (1992); Wiman, et al., Thrombosis and Haemostasis, 74, 71, (1995); V. Salomaa, et al., Circulation, 91, 284 (1995); and Eitzman, Blood, 96, 4212 (2000). In animal experiments, inhibitors of PAI-1 activity have been shown to be effective at treating thrombotic conditions. See e.g., Berry, et al, British Journal of Pharmacology, 125, 29 (1998) and Friedrich, et al., Circulation, 96, 916, (1997).

In tissue matrices, u-PA converts plasminogen to plasmin which activates matrix metalloproteases (MMPs) that degrade extracellular matrix (ECM). Through this regulation of u-PA, PAI-1 therefore plays an important role in cellular migration and tissue remodelling processes. PAI-1 is thus believed to modulate diseases and conditions such as wound healing, angiogenesis, cancer invasion, and metastasis. Increased levels of PAI-1 have been associated with poor prognosis in cancer patients (T. L. Frandesen, Drugs Future, 873, 1998; Pappot, et al, Biol. Chem. Hoppe-Seyler, 376, 259, 1995). PAI-1 has additionally been associated with other conditions and diseases such as obesity and insulin resistance (Juhan-Vague, et al, Journal of Thrombosis and Haemostasis, 1, 1575, 2003), inflammatory diseases, such as asthma (Cho, et al, Journal of Allergy and Clinical Immunology, 108, 212, 2001), and renal disease (Brown, et al, Journal of Nephrology, 15, 230, 2002). See also Tsikouris et al., J. Clin. Pharmacol., 42:1187, 2002 and Binder et al., News Physiol. Sci. 17, 56, 2002.

Accordingly, compounds that inhibit PAI-1 would be useful in the treatment of several disease states and disorders, especially thromboembolic disorders.

### SUMMARY OF THE INVENTION

The instant invention pertains to methods of inhibiting PAI-1 inhibitors comprising administering to a patient in need thereof a therapeutically effective amount of at least one compound of formula (I), at least one compound of formula (I), 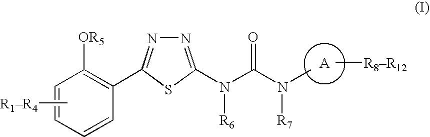
or a pharmaceutically-acceptable salt, prodrug, stereoisomer or solvate thereof, wherein:
A is aryl or heteroaryl;
R₁-R₄ and R₈-R₁₂ are independently selected from hydrogen, halogen, nitro, cyano, alkyl, substituted alkyl, aryl, heteroaryl, cycloalkyl, heterocyclo, —OR₁₃, —SR₁₄, —OC(═O)R₁₃, —C(═O)R₁₃, —CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅;
or any two of R₁-R₄ and R₈-R₁₂ located on neighboring atoms of the ring to which they are attached may be taken together to form a fused ring system in combination with the ring, wherein the fused ring system may be optionally further substituted;
R₅ is hydrogen, alkyl or substituted alkyl (preferably hydrogen, methyl or CF₃, more preferably hydrogen);
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and/or R₁₆ is selected independently.

The invention further relates to compounds having formula (Ia), 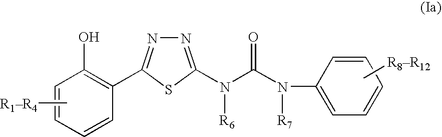
or a pharmaceutically-acceptable salt, prodrug, stereoisomer or solvate thereof, wherein:
R₁-R₄ and R₈-R₁₂ are independently selected from hydrogen, halogen, nitro and cyano, alkyl, substituted alkyl, aryl, heteroaryl, cycloalkyl, heterocyclo, —OR₁₃, —SR₁₃, —OC(═O)R₁₃, —C(═O)R₁₃—CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, —N₁₆C(═O)NR₁₄R₁₅; provided that when any one of R₈-R₁₂ is —N₁₆C(═O)NR₁₄R₁₅, neither R₁₄ or R₁₅ are thiadiazole;
or any two of R₁-R₄ and R₈-R₁₂ located on neighboring atoms of the ring to which they are attached may be taken together to form a fused ring system in combination with the ring, wherein the fused ring system may be optionally further substituted;
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and R₁₆ is selected independently.

The invention also pertains to pharmaceutical compositions within the scope of formula (I) as well as medicaments and articles of manufacture comprising compounds of formula (I).

### DESCRIPTION OF THE INVENTION

Listed below are definitions of various terms used to describe this invention. These definitions apply to the terms as they are used throughout this specification, unless otherwise limited in specific instances, either individually or as part of a larger group.

The term “alkyl” refers to straight or branched chain unsubstituted hydrocarbon groups of 1 to 20 carbon atoms, preferably 1 to 7 carbon atoms. The expression “lower alkyl” refers to unsubstituted alkyl groups of 1 to 4 carbon atoms. When a subscript is used with reference to an alkyl or other group, the subscript refers to the number of carbon atoms that the group may contain. For example, the term “C₀₋₄alkyl” includes a bond and alkyl groups of 1 to 4 carbon atoms.

The term “substituted alkyl” refers to an alkyl group substituted by one to four substituents selected from halogen, hydroxy, alkoxy, keto (═O), alkanoyl, aryloxy, alkanoyloxy, NRₐR_{b}, alkanoylamino, aroylamino, aralkanoylamino, substituted alkanoylamino, substituted arylamino; substituted aralkanoylamino, thiol, alkylthio, arylthio, aralkylthio, alkylthiono, arylthiono, aralkylthiono, alkylsulfonyl, arylsulfonyl, aralkylsulfonyl, —SO₂NRₐR_{b}, nitro, cyano, —CO₂H, —CONRₐR_{b}, alkoxycarbonyl, aryl, guanidino and heteroaryls or heterocyclos (such as indolyl, imidazolyl, furyl, thienyl, thiazolyl, pyrrolidyl, pyridyl, pyrimidyl and the like), wherein Rₐ and R_{b} are selected from hydrogen, alkyl, aryl, aralkyl, cycloalkyl, cycloalkylalkyl, heteroaryl, heteroarylalkyl, heterocycle, and heterocyclealkyl. The substituent on the alkyl optionally in turn may be further substituted, in which case it will be with substituted one or more of C₁₋₄alkyl, C₂₋₄alkenyl, halogen, haloalkyl, haloalkoxy, cyano, nitro, amino, C₁₋₄alkylamino, aminoC₁₋₄alkyl, hydroxy, hydroxyC₁₋₄alkyl, alkoxy, alkylthio, phenyl, benzyl, phenyloxy, and/or benzyloxy.

The term “alkenyl” refers to straight or branched chain hydrocarbon groups of 2 to 20 carbon atoms, preferably 2 to 15 carbon atoms, and most preferably 2 to 8 carbon atoms, having at least one double bond, and depending on the number of carbon atoms, up to four double bonds.

The term “substituted alkenyl” refers to an alkenyl group substituted by one to two substituents selected from those recited above for substituted alkyl groups.

The term “alkynyl” refers to straight or branched chain hydrocarbon groups of 2 to 20 carbon atoms, preferably 2 to 15 carbon atoms, and most preferably 2 to 8 carbon atoms, having at least one triple bond, and depending on the number of carbon atoms, up to four triple bonds.

The term “substituted alkynyl” refers to an alkynyl group substituted by one to two substituents selected from those recited above for alkyl groups.

When the term alkyl is used in connection with another group, as in heterocycloalkyl or cycloalkylalkyl, this means the identified (first named) group is bonded directly through an alkyl group which may be branched or straight chain (e.g., cyclopropylC₁₋₄alkyl means a cyclopropyl group bonded through a straight or branched chain alkyl group having one to four carbon atoms.). In the case of substituents, as in “substituted cycloalkylalkyl,” the alkyl portion of the group, besides being branched or straight chain, may be substituted as recited above for substituted alkyl groups and/or the first named group (e.g., cycloalkyl) may be substituted as recited herein for that group.

The term “halogen” or “halo” refers to fluorine, chlorine, bromine and iodine.

The term “aryl” refers to monocyclic, bicyclic or tricyclic aromatic substituted or unsubstituted hydrocarbon groups having 6 to 12 carbon atoms in the ring portion, such as phenyl, naphthyl, and biphenyl groups. Each ring of the aryl may be optionally substituted with one to three R_{c} groups, wherein R_{c} at each occurrence is selected from alkyl, substituted alkyl, halogen, trifluoromethoxy, trifluoromethyl, —SR, —OR, —NRR′, —NRSO₂R′, —SO₂R, —SO₂NRR′, —CO₂R′, —C(═O)R′, —C(═O)NRR′, —OC(═O)R′, —OC(═O)NRR′, —NRC(═O)R′, —NRCO₂R′, phenyl, C₃₋₇ cycloalkyl, and five-to-six membered heterocyclo or heteroaryl, wherein each R and R′ is selected from hydrogen, alkyl, substituted alkyl, alkenyl, substituted alkenyl, phenyl, C₃₋₇cycloalkyl, and five-to-six membered heterocyclo or heteroaryl, except in the case of a sulfonyl group, then R is not going to be hydrogen. Each substituent R_{c} optionally in turn may be further substituted by one or more (preferably 0 to 2) R_{d} groups, wherein R_{d} is selected from C₁₋₆alkyl, C₂₋₆alkenyl, halogen, haloalkyl, haloalkoxy, cyano, nitro, amino, C₁₋₄alkylamino, aminoC₁₋₄alkyl, hydroxy, hydroxyC₁₋₄alkyl, alkoxy, alkylthio, phenyl, benzyl, phenylethyl, phenyloxy, and benzyloxy.

Exemplary fused ring systems especially fused phenyl groups include, but are not limited to, the following core groups, all of which may be substituted as described above:

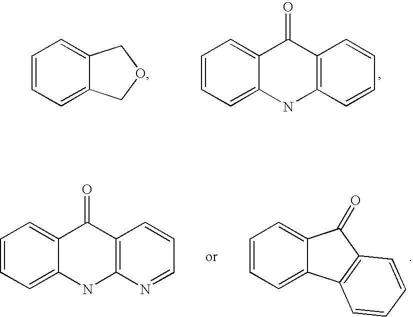

The term “aralkyl” refers to an aryl group bonded directly through an alkyl group, such as benzyl, wherein the alkyl group may be branched or straight chain. In the case of a “substituted aralkyl,” the alkyl portion of the group besides being branched or straight chain, may be substituted as recited above for substituted alkyl groups and/or the aryl portion may be substituted as recited herein for aryl. Thus, the term “optionally substituted benzyl” refers to the group 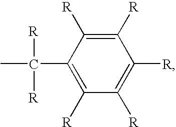
wherein each R group may be hydrogen or may also be selected from R_{c} as defined above, in turn optionally substituted with one or more R_{d}. At least two of these “R” groups should be hydrogen and preferably at least five of the “R” groups is hydrogen. A preferred benzyl group involves the alkyl-portion being branched to define

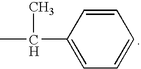

The term “heteroaryl” refers to a substituted or unsubstituted aromatic group for example, which is a 4 to 7 membered monocyclic, 7 to 11 membered bicyclic, or 10 to 15 membered tricyclic ring system, which has at least one heteroatom and at least one carbon atom-containing ring. Each ring of the heteroaryl group containing a heteroatom can contain one or two oxygen or sulfur atoms and/or from one to four nitrogen atoms, provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. It may optionally be substituted with one to three (preferably 0 to 2) R_{c} groups, as defined above for aryl, which in turn may be substituted with one or more (preferably 0 to 2) R_{d} groups, also as recited above.

Exemplary monocyclic heteroaryl groups include pyrrolyl, pyrazolyl, pyrazolinyl, imidazolyl, oxazolyl, isoxazolyl, thiazolyl 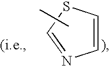
thiadiazolyl, isothiazolyl, furanyl, thienyl, oxadiazolyl, pyridyl, pyrazinyl, pyrimidinyl, pyridazinyl, triazinyl and the like, especially pyridyl, thienyl, pyrrolyl, imidazolyl, triazolyl, tetrazolyl, pyrazolyl and furyl.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like, especially indolyl, benzylmidazolyl, benzyopyranyl.

Exemplary tricyclic heteroaryl groups include carbazolyl, benzidolyl, phenanthrollinyl, acridinyl, phenanthridinyl, xanthenyl and the like.

The term “cycloalkyl” refers to a saturated or partially unsaturated non-aromatic cyclic hydrocarbon ring system, preferably containing 1 to 3 rings and 3 to 7 carbon atoms per ring, which may be substituted or unsubstituted and/or which may be fused with a C₃-C₇ carbocylic ring, a heterocyclic ring, or which may have a bridge of 3 to 4 carbon atoms. The cycloalkyl groups including any available carbon or nitrogen atoms on any fused or bridged rings optionally may have 0 to 3 (preferably 0-2) substituents selected from R_{c} groups, as recited above, and/or from keto (where appropriate) which in turn may be substituted with one to three R_{d} groups, also as recited above. Thus, when it is stated that a carbon-carbon bridge may be optionally substituted, it is meant that the carbon atoms in the bridged ring optionally may be substituted with an R_{c} group, which preferably is seleted from C₁₋₄alkyl, C₂₋₄alkenyl, halogen, haloalkyl, haloalkoxy, cyano, amino, C₁₋₄alkylamino, aminoC₁₋₄alkyl, hydroxy, hydroxyC₁₋₄alkyl, and C₁₋₄alkoxy. Exemplary groups include cyclopropyl, cyclobutyl, cyclopentyl, cyclohexyl, cycloheptyl, bicycloheptane, cycloctyl, cyclodecyl, cyclododecyl, and adamantyl.

The terms “heterocycle”, “heterocyclic” and “heterocyclo” each refer to a fully saturated or partially unsaturated nonaromatic cyclic group, which may be substituted or unsubstituted, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen, oxygen, and sulfur atoms, where the nitrogen and sulfur heteroatoms also optionally may be oxidized and the nitrogen heteroatoms also optionally may be quaternized. Preferably two adjacent heteroatoms are not simultaneously selected from oxygen and nitrogen. The heterocyclic group may be attached at any nitrogen or carbon atom. The heterocyclo groups optionally may have 0 to 3 (preferably 0-2) substituents selected from keto (═O), and/or one or more R_{c} groups, as recited above, which in turn may be substituted with one to three Rd groups, also as recited above.

Exemplary monocyclic heterocyclic groups include pyrrolidinyl, pyrrolyl, indolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, dioxanyl, isothiazolidinyl, thietanyl, thiiranyl, triazinyl, and triazolyl, and the like.

Exemplary bicyclic hetrocyclic groups include 2,3-dihydro-2-oxo-1H-indolyl, benzothiazolyl, benzoxazolyl, benzothienyl, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl (such as furo[2,3-c]pyridinyl, furo[3,1-b]pyridinyl] or furo[2,3-b]pyridinyl), dihydroisoindolyl, dihydroquinazolinyl (such as 3,4-dihydro-4-oxo-quinazolinyl), benzisothiazolyl, benzisoxazolyl, benzodiazinyl, benzofurazany, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, thienothienyl, and the like, especially.

Also included are smaller heterocyclos, such as epoxides and aziridines.

Unless otherwise indicated, when reference is made to a specifically-named aryl (e.g., phenyl), cycloalkyl (e.g., cyclohexyl), heterocyclo (e.g., pyrrolidinyl) or heteroaryl (e.g., indolyl), the reference is intended to include rings having 0 to 3, preferably 0-2, substituents selected from those recited above for the the aryl, cycloalkyl, heterocyclo and/or heteroaryl groups, as appropriate. Additionally, when reference is made to a specific heteroaryl or heterocyclo group, the reference is intended to include those systems having the maximum number of non-cumulative double bonds or less than the maximum number of double bonds. Thus, for example, the term “isoquinoline” refers to isoquinoline and tetrahydroisoquinoline.

Additionally, it should be understood that one skilled in the field may make appropriate selections for the substituents for the aryl, cycloalkyl, heterocyclo, and heteroaryl groups to provide stable compounds and compounds useful as pharmaceutically-acceptable compounds and/or intermediate compounds useful in making pharmaceutically-acceptable compounds. Thus, for example, in compounds of formula (I), when a substituent is a cyclopropyl ring, preferably the ring has no more than two substituents, and preferably said substituents do not comprise nitro (NO₂), more than one cyano group, or three halogen groups. Similarly, when m is 3, preferably R₆, the substituents on the phenyl ring A, are not all nitro, and so forth.

The term “heteroatoms” shall include oxygen, sulfur and nitrogen.

The term “haloalkyl” means an alkyl having one or more halo substituents.

The term “perfluoromethyl” means a methyl group substituted by one, two, or three fluoro atoms, i.e., CH₂F, CHF₂ and CF₃. The term “perfluoroalkyl” means an alkyl group having from one to five fluoro atoms, such as pentafluoroethyl.

The term “haloalkoxy” means an alkoxy group having one or more halo substituents. For example, “haloalkoxy” includes —OCF₃.

The term “carbocyclic” means a saturated or unsaturated monocyclic or bicyclic ring in which all atoms of all rings are carbon. Thus, the term includes cycloalkyl and aryl rings. The carbocyclic ring may be substituted in which case the substituents are selected from those recited above for cycloalkyl and aryl groups.

When the term “unsaturated” is used herein to refer to a ring or group, the ring or group may be fully unsaturated or partially unsaturated.

Definitions for the various other groups that are recited above in connection with substituted alkyl, substituted alkenyl, aryl, cycloalkyl, and so forth, are as follows: alkoxy is —OR^{e}, alkanoyl is —C(═O)R^{e}, aryloxy is —OAr, alkanoyloxy is —OC(═O)R^{e}, amino is —NH₂, alkylamino is —NHR^{e} or —N(Re)₂, arylamino is —NHAr or —NR^{e}Ar, aralkylamino is —NH—R^{f}—Ar, alkanoylamino is —NH—C(═O)Re, aroylamino is —NH—C(═O)Ar, aralkanoylamino is —NH—C(═O)R^{f}—Ar, thiol is —SH, alkylthio is —SR^{e}, arylthio is —SAr, aralkylthio is —S-R^{f}-Ar, alkylthiono is —S(═O)Re, arylthiono is —S(═O)Ar, aralkylthiono is —S(═O)R^{f}—Ar, alkylsulfonyl is —SO_{(q)}R^{e}, arylsulfonyl is —SO_{(q)}Ar, arylsulfonylamine is —NHSO_{(q)}Ar, alkylsulfonylamine is —NHSO₂R^{e}, aralkylsulfonyl is —SO_{(q)}R^{f}Ar, sulfonamido is —SO₂NH₂, substituted sulfonamide is —SO₂NHR^{e} or —SO₂N(R^{e})₂, nitro is —NO₂, carboxy is —CO₂H, carbamyl is —CONH₂, substituted carbamyl is —C(═O)NHR^{g} or —C(═O)NR^{g}R^{h}, alkoxycarbonyl is —C(═O)OR^{e}, carboxyalkyl is —R^{f}—CO₂H, sulfonic acid is —SO₃H, arylsulfonylamine is —NHSO_{(q)}Ar, guanidino is 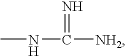
and ureido is 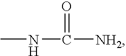
wherein R^{e} is alkyl or substituted alkyl as defined above, R^{f} is alkylene or substituted alkylene as defined above, R^{g} and R^{h} are selected from alkyl, substituted alkyl, aryl, aralkyl, cycloalkyl, heterocyclo, and heteraryl; Ar is an aryl as defined above, and q is 2 or 3.

Throughout the specification, groups and substituents thereof may be chosen by one skilled in the field to provide stable moieties and compounds.

The compounds of Formula (I) may form salts which are also within the scope of this invention. Pharmaceutically acceptable (i.e. non-toxic, physiologically acceptable) salts are preferred, although other salts are also useful, e.g., in isolating or purifying the compounds of this invention.

The compounds of Formula (I) may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds for Formula (I) may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art. Salt forms of the compounds may be advantageous for improving the compound dissolution rate and oral bioavailability.

In addition, zwitterions (“inner salts”) may be formed.

All stereoisomers of the compounds of the instant invention are contemplated, either in admixture or in pure or substantially pure form. The definition of compounds according to the invention embraces all the possible stereoisomers and their mixtures. It embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

Compounds of the Formula (I) may also have prodrug forms. Any compound that will be converted in vivo to provide the bioactive agent (i.e., the compound for formula I) is a prodrug within the scope and spirit of the invention.

Various forms of prodrugs are well known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Acamedic Press, 1985);
b) A Textbook of Drug Design and Development, edited by Krosgaard-Larsen and H. Bundgaard, Chapter 5, “Design and Application of Prodrugs,” by H. Bundgaard, p. 113-191 (1991); and
c) H. Bundgaard, Advanced Drug Delivery Reviews, 8, 1-38 (1992), each of which is incorporated herein by reference.

It should further be understood that solvates (e.g., hydrates) of the compounds of Formula (I) are also with the scope of the present invention. Methods of solvation are generally known in the art.

### Preferred Methods and Compounds

Preferred methods of treating a disorder associated with high levels of PAI-1 are those comprising administering an to patient in need of such treatment an effective amount of at least one compound having the formula (Ia), 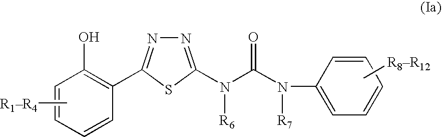
or a pharmaceutically-acceptable salt, prodrug, stereoisomer or solvate thereof, wherein:
R₁-R₄ and R₈-R₁₂ are independently selected from hydrogen, halogen, nitro and cyano, alkyl, substituted alkyl, aryl, heteroaryl, cycloalkyl, heterocyclo, —OR₁₃, —SR₁₃, —OC(═O)R₁₃, —C(═O)R₁₃—CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, —N₁₆C(═O)NR₁₄R₁₅; provided that when any one of R₈-R₁₂ is —NR₁₆C(═O)NR₁₄R₁₅, neither R₁₄ or R₁₅ are thiadiazole;
or any two of R₁-R₄ and R₈-R₁₂ located on neighboring atoms of the ring to which they are attached may be taken together to form a fused ring system in combination with the ring, wherein the fused ring system may be optionally further substituted;
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and R₁₆ is selected independently.

More preferred methods of treating a disorder associated with high levels of PAI-1 are those comprising administering to a patient in need of such treatment an effective amount of at least one compound within the scope of formula (I) having the formula (Ia), 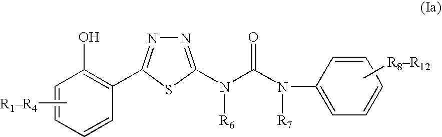
or a pharmaceutically-acceptable salt, prodrug, stereoisomer or solvate thereof, wherein:
R₁-R₄ and R₈-R₁₂ are independently selected from hydrogen, halogen, nitro, cyano, alkyl, substituted alkyl, cycloalkyl, aryl, heteroaryl, heterocyclo, —OR₁₃, —SR₁₄, —OC(═O)R₁₃, —CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃ and —NR₁₆C(═O)NR₁₄R₁₅);
or any two of R₁-R₅ and R₈-R₁₂ located on neighboring atoms of the ring to which they are attached may be taken together to form a fused ring system in combination with the ring, wherein the fused ring system may be optionally further substituted;
   Even more preferred methods comprising administering to a patient in need thereof a therapeutically effective amount of at least one compound within the scope of formula (Ia) in which at least one and preferably all of the variables are chosen as below:
R₁-R₄ are independently selected from hydrogen, halogen, nitro, cyano, alkyl, substituted alkyl, —OR₁₃ and —SR₁₃; and
R₈-R₁₂ are independently selected from hydrogen, halogen, nitro, cyano, alkyl, substituted alkyl, aryl, heteroaryl, cycloalkyl, heterocyclo, —OR₁₃, —SR₁₃, —OC(═O)R₁₃, —C(═O)R₁₃, —CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅, provided that when any one of R₈-R₁₂ is —NR₁₆C(═O)NR₁₄R₁₅, neither R₁₄ or R₁₅ are thiadiazole;
or any two of R₈-R₁₂ located on neighboring atoms of the ring may be taken together to form a fused ring system in combination with the ring, said fused ring system being optionally substituted.
   Especially preferred of the aforementioned methods are those in which the variables R₈-R₁₂ are selected from:
   (a) hydrogen, nitro, cyano, halogen, —OR₁₃, —C(═O)R₁₃, —CO₂R₁₃, —C(═O)NR₁₄R₁₅, —SR₁₃, —S(═O)R₁₃, —SO₂R₁₃, and —SO₂NR₁₄R₁₅; or
   (b) C₁₋₆alkyl, substituted C₁₋₆alkyl, heterocyclo and heteroaryl; or
   (c) any two of R₈-R₁₂ located on neighboring atoms of the ring may be taken together to form a fused ring system in combination with the ring, said fused ring system being optionally substituted.

Even more preferred methods within the scope of formula (Ia) are those comprising administering to a patient in need thereof a therapeutically effective amount of at least one compound having formula (Ib) 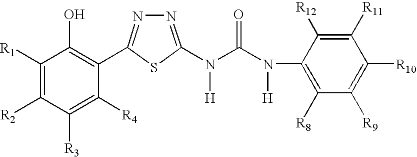
or a pharmaceutically-acceptable salt, prodrug, stereoisomer or solvate thereof, wherein:
R₁-R₄ are selected from hydrogen, halogen, nitro and C₁₋₆alkoxy, especially wherein:
R₁ and R₃ are halogen; and R₂ and R₄ are hydrogen.

Preferred compounds are those having the formula (Ia), 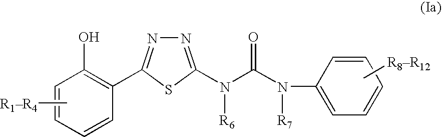
or a pharmaceutically-acceptable salt, prodrug, stereoisomer or solvate thereof, wherein:
R₁-R₄ and R₈-R₁₂ are independently selected from hydrogen, halogen, nitro and cyano, alkyl, substituted alkyl, aryl, heteroaryl, cycloalkyl, heterocyclo, —OR₁₃, —SR₁₃, —OC(═O)R₁₃, —C(═O)R₁₃—CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, —N₁₆C(═O)NR₁₄R₁₅; provided that when any one of R₈-R₁₂ is —NR₁₆C(═O)NR₁₄R₁₅, neither R₁₄ or R₁₅ are thiadiazole;
or any two of R₁-R₄ and R₈-R₁₂ located on neighboring atoms of the ring to which they are attached may be taken together to form a fused ring system in combination with the ring, wherein the fused ring system may be optionally further substituted;
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and R₁₆ is selected independently.

Even more preferred compounds within the scope of formula (Ia) are those in which at least one variable, preferably all, are chosen from:
R₁-R₄ are independently selected from hydrogen, halogen, nitro, cyano, alkyl, substituted alkyl, —OR₁₃ and —SR₁₃; and
R₈-R₁₂ are independently selected from hydrogen, halogen, nitro, cyano, alkyl, substituted alkyl, aryl, heteroaryl, cycloalkyl, heterocyclo, —OR₁₃, —SR₁₃, —OC(═O)R₁₃, —C(═O)R₁₃, —CO₂R₁₃, —C(═O)NR₁₄R₁₅, —NR₁₄R₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅, provided that when any one of R₈′-R₁₂ is —NR₁₆C(═O)NR₁₄R₁₅, neither R₁₄ or R₁₅ are thiadiazole
or any two of R₈-R₁₂ located on neighboring atoms of the ring may be taken together to form a fused ring system in combination with the ring, said fused ring system being optionally substituted.
   Especially preferred compounds within the immediately aforementioned scope are those in which R₈-R₁₂ are selected from
   (a) hydrogen, nitro, cyano, halogen, —OR₁₃, —C(═O)R₁₃, —CO₂R₁₃, —C(═O)NR₁₄R₁₅, —SR₁₃, —S(═O)R₁₃, —SO₂R₁₃, and —SO₂NR₁₄R₁₅; or
   (b) C₁₋₆alkyl, substituted C₁₋₆alkyl, heterocyclo and heteroaryl; or
   (c) any two of R₈-R₁₂ located on neighboring atoms of the ring may be taken together to form a fused ring system in combination with the ring, said fused ring system being optionally substituted.

Even more preferred compounds within the scope of formula (Ia) are those having formula (Ib) 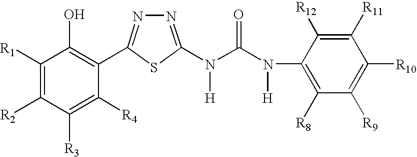
or a pharmaceutically-acceptable salt, prodrug, stereoisomer or solvate thereof, in which one of the variables, and preferably all are selected from below:
R₁-R₄ are selected from hydrogen, halogen, nitro and C₁₋₆alkoxy, and
R₉ and R₁₀ are taken together to form a fused ring system in combination with the phenyl ring, said fused ring system substituted where valence allows with a group selected from hydrogen, oxo, nitro, cyano, halogen, C₁₋₆alkyl, substituted C₁₋₆alkyl, C₁₋₆alkyloxy and C₁₋₆haloalkyloxy.

Alternatively even more preferred compounds within the scope of formula (Ib), or a pharmaceutically-acceptable salt, prodrug, stereoisomer or solvate thereof, are those in which at least one of the variables, and preferably all, are selected from below:
R₁-R₄ are selected from hydrogen, halogen, nitro and C₁₋₆alkoxy, and
R₁₀ is —C(═O)R₁₃; and
R₁₃ is aryl (especially phenyl) or heteroaryl (especially pyridinyl, thienyl, and pyrrolyl) substituted with a group selected from hydrogen, nitro, cyano, halogen, aryl, heteroaryl, C₁₋₆alkyl, substituted C₁₋₆alkyl, C₁₋₆alkyloxy and C₁₋₆haloalkyloxy, C(═O)R₁₇, CO₂R₁₇, NR₁₇R₁₈, and —OR₁₇; and
R₁₇ and R₁₈ are independently hydrogen, C₁₋₆alkyl or phenyl.

Alternatively even more preferred compounds within the scope of formula (Ib), or a pharmaceutically-acceptable salt, prodrug, stereoisomer or solvate thereof, are those in which at least one of the variables, and preferably all, are selected from below:
R₁-R₄ are selected from hydrogen, halogen, nitro and C₁₋₆alkoxy, and
R₁₁ is —C(═O)NR₁₄R₁₅;
R₁₄ is hydrogen or C₁₋₆alkyl;
R₁₅ is aryl or heteroaryl substituted with a group selected from hydrogen, amino, nitro, cyano, halogen, aryl, heteroaryl, C₁₋₆alkyl, substituted C₁₋₆alkyl, C₁₋₆alkyloxy and C₁₋₆haloalkyloxy, C(═O)R₁₇, CO₂R₁₇, and —OR₁₇; and
R₁₇ and R₁₈ are independently hydrogen, C₁₋₆alkyl or phenyl.

Also preferred compounds within the scope of formula (Ib), or a pharmaceutically-acceptable salt, prodrug, stereoisomer or solvate thereof, are those in which:
R₁ and R₃ are halogen; and
R₂ and R₄ are hydrogen.

Other preferred compounds are selected from:
(i) 1-[5-(3,5-Dichloro-2-hydroxy-phenyl)-[1,3,4]thiadiazol-2-yl]-3-(4-nitro-phenyl)-urea;
4-{3-[5-(3,5-Dichloro-2-hydroxy-phenyl)-[1,3,4]thiadiazol-2-yl]-ureido}-benzoic acid ethyl ester;
3-{3-[5-(3,5-Dichloro-2-hydroxy-phenyl)-[1,3,4]thiadiazol-2-yl]-ureido}-benzoic acid methyl ester;
4-{3-[5-(3,5-Dichloro-2-hydroxy-phenyl)-[1,3,4]thiadiazol-2-yl]-ureido}-benzoic acid methyl ester;
3-{3-[5-(3,5-Dichloro-2-hydroxy-phenyl)-[1,3,4] thiadiazol-2-yl]-ureido}-benzoic acid;
3-{3-[5-(3,5-Dichloro-2-hydroxy-phenyl)-[1,3,4]thiadiazol-2-yl]-ureido}-N-phenyl-benzamide;
3-{3-[5-(3,5-Dichloro-2-hydroxy-phenyl)-[1,3,4]thiadiazol-2-yl]-ureido}-N-(4-dimethylamino-phenyl)-benzamide;
3-{3-[5-(3,5-Dichloro-2-hydroxy-phenyl)-[1,3,4]thiadiazol-2-yl]-ureido}-N-(3-hydroxymethyl-phenyl)-benzamide;
3-{3-[5-(3,5-Dichloro-2-hydroxy-phenyl)-[1,3,4]thiadiazol-2-yl]-ureido}-N-(2-methoxy-phenyl)-benzamide;
3-{3-[5-(3,5-Dichloro-2-hydroxy-phenyl)-[1,3,4]thiadiazol-2-yl]-ureido}-N-(4-methoxy-phenyl)-benzamide;
3-{3-[5-(3,5-Dichloro-2-hydroxy-phenyl)-[1,3,4]thiadiazol-2-yl]-ureido}-N-m-tolyl-benzamide;
3-{3-[5-(3,5-Dichloro-2-hydroxy-phenyl)-[1,3,4]thiadiazol-2-yl]-ureido}-N-(4-pentyloxy-phenyl)-benzamide;
3-{3-[5-(3,5-Dichloro-2-hydroxy-phenyl)-[1,3,4]thiadiazol-2-yl]-ureido}-N-(1H-indol-5-yl)-benzamide;
3-{3-[5-(3,5-Dichloro-2-hydroxy-phenyl)-[1,3,4]thiadiazol-2-yl]-ureido}-N-(4-phenoxy-phenyl)-benzamide;
1-[5-(3,5-Dichloro-2-hydroxy-phenyl)-[1,3,4]thiadiazol-2-yl]-3-(3-oxo-1,3-dihydro-isobenzofuran-5-yl)-urea;
1-(2-Benzoyl-4-nitro-phenyl)-3-[5-(3,5-dichloro-2-hydroxy-phenyl)-[1,3,4]thiadiazol-2-yl]-urea;
1-[5-(3,5-Dichloro-2-hydroxy-phenyl)-[1,3,4]thiadiazol-2-yl]-3-(4-hydroxy-phenyl)-urea;
1-(4-Benzotriazol-2-yl-phenyl)-3-[5-(3,5-dichloro-2-hydroxy-phenyl)-[1,3,4]thiadiazol-2-yl]-urea;
1-[4-(4-Bromo-1-methyl-1H-pyrazol-3-yl)-phenyl]-3-[5-(3,5-dichloro-2-hydroxy-phenyl)-[1,3,4] thiadiazol-2-yl]-urea;
1-[5-(3,5-Dichloro-2-hydroxy-phenyl)-[1,3,4]thiadiazol-2-yl]-3-(4-imidazol-1-yl-phenyl)-urea;
5-(4-{3-[5-(3,5-Dichloro-2-hydroxy-phenyl)-[1,3,4]thiadiazol-2-yl]-ureido}-phenyl)-4-methyl-furan-3-carboxylic acid ethyl ester;
1-[5-(3,5-Dichloro-2-hydroxy-phenyl)-[1,3,4]thiadiazol-2-yl]-3-(4-[1,2,4]triazol-1-yl-phenyl)-urea;
1-[5-(3,5-Dichloro-2-hydroxy-phenyl)-[1,3,4]thiadiazol-2-yl]-3-(9-oxo-9H-fluoren-3-yl)-urea;
1-[5-(3,5-Dichloro-2-hydroxy-phenyl)-[1,3,4] thiadiazol-2-yl]-3-[4-(4-methoxy-benzoyl)-phenyl]-urea;
1-[5-(3,5-Dichloro-2-hydroxy-phenyl)-[1,3,4]thiadiazol-2-yl]-3-(1-oxo-1,3-dihydro-isobenzofuran-5-yl)-urea;
1-[5-(3,5-Dichloro-2-hydroxy-phenyl)-[1,3,4]thiadiazol-2-yl]-3-(2-methoxy-5-nitro-phenyl)-urea;
1-[5-(3,5-Dichloro-2-hydroxy-phenyl)-[1,3,4]thiadiazol-2-yl]-3-(2-fluoro-5-nitro-phenyl)-urea;
4-{3-[5-(3,5-Dichloro-2-hydroxy-phenyl)-[1,3,4]thiadiazol-2-yl]-ureido}-3-methyl-benzoic acid ethyl ester;
1-(4-Benzoyl-phenyl)-3-[5-(3,5-dichloro-2-hydroxy-phenyl)-[1,3,4]thiadiazol-2-yl]-urea;
1-[5-(3,5-Dichloro-2-hydroxy-phenyl)-[1,3,4]thiadiazol-2-yl]-3-[4-(pyridine-4-carbonyl)-phenyl]-urea;
Hydrochloride of 1-[5-(3,5-Dichloro-2-hydroxy-phenyl)-[1,3,4]thiadiazol-2-yl]-3-[4-(pyridine-3-carbonyl)-phenyl]-urea;
1-[5-(3,5-Dichloro-2-hydroxy-phenyl)-[1,3,4]thiadiazol-2-yl]-3-[4-(4-methyl-benzoyl)-phenyl]-urea;
1-[5-(3,5-Dichloro-2-hydroxy-phenyl)-[1,3,4]thiadiazol-2-yl]-3-[4-(4-fluoro-benzoyl)-phenyl]-urea;
1-[5-(3,5-Dichloro-2-hydroxy-phenyl)-[1,3,4]thiadiazol-2-yl]-3-[4-(2-fluoro-benzoyl)-phenyl]-urea;
1-[5-(3,5-Dichloro-2-hydroxy-phenyl)-[1,3,4]thiadiazol-2-yl]-3-(9-oxo-9,10-dihydro-acridin-3-yl)-urea;
1-[5-(3,5-Dichloro-2-hydroxy-phenyl)-[1,3,4]thiadiazol-2-yl]-3-(10-methyl-9-oxo-9,10-dihydro-acridin-3-yl)-urea;
1-[5-(3,5-Dichloro-2-hydroxy-phenyl)-[1,3,4]thiadiazol-2-yl]-3-(5-oxo-5, 10-dihydro-benzo[b] [1,8]naphthyridin-8-yl)-urea;
1-[5-(3,5-Dichloro-2-hydroxy-phenyl)-[1,3,4]thiadiazol-2-yl]-3-[4-(thiophene-2-carbonyl)-phenyl]-urea;
(3-{3-[5-(3,5-Dichloro-2-hydroxy-phenyl)-[1,3,4]thiadiazol-2-yl]-ureido}-9-oxo-9H-acridin-10-yl)-acetic acid ethyl ester;
1-[5-(3,5-Dichloro-2-hydroxy-phenyl)-[1,3,4]thiadiazol-2-yl]-3-[4-(1H-pyrrole-2-carbonyl)-phenyl]-urea;
1-[5-(3,5-Dichloro-2-hydroxy-phenyl)-[1,3,4]thiadiazol-2-yl]-3-[4-(1-methyl-1H-pyrrole-2-carbonyl)-phenyl]-urea;
4-(4-{3-[5-(3,5-Dichloro-2-hydroxy-phenyl)-[1,3,4]thiadiazol-2-yl]-ureido}-benzoyl)-benzoic acid ethyl ester;
3-{3-[5-(3,5-Dichloro-2-hydroxy-phenyl)-[1,3,4]thiadiazol-2-yl]-ureido}-N-methyl-N-phenyl-benzamide;
N-Butyl-3-{3-[5-(3,5-dichloro-2-hydroxy-phenyl)-[1,3,4]thiadiazol-2-yl]-ureido}-N-phenyl-benzamide;
1-[5-(3,5-Dichloro-2-hydroxy-phenyl)-[1,3,4]thiadiazol-2-yl]-3-[4-(2-methyl-2H-tetrazol-5-yl)-phenyl]-urea;
1-[5-(3,5-Dichloro-2-hydroxy-phenyl)-[1,3,4]thiadiazol-2-yl]-3-[4-(4-dimethylamino-benzoyl)-phenyl]-urea;
1-[5-(3,5-Dichloro-2-hydroxy-phenyl)-[1,3,4]thiadiazol-2-yl]-3-[4-(4-[1,2,3]triazol-2-yl-benzoyl)-phenyl]-urea;
1-[5-(3,5-Dichloro-2-hydroxy-phenyl)-[1,3,4]thiadiazol-2-yl]-3-[4-(4-[1,2,3]triazol-1-yl-benzoyl)-phenyl]-urea;
1-[5-(3,5-Dichloro-2-hydroxy-phenyl)-[1,3,4]thiadiazol-2-yl]-3-[4-(4-[1,2,4]triazol-1-yl-benzoyl)-phenyl]-urea;
1-[5-(3,5-Dichloro-2-hydroxy-phenyl)-[1,3,4]thiadiazol-2-yl]-3-[4-(4-imidazol-1-yl-benzoyl)-phenyl]-urea;
1-[5-(3,5-Dichloro-2-hydroxy-phenyl)-[1,3,4] thiadiazol-2-yl]-3-[4-(pyrrole-1-carbonyl)-phenyl]-urea;
1-[4-(2-Acetyl-pyrrole-1-carbonyl)-phenyl]-3-[5-(3,5-dichloro-2-hydroxy-phenyl)-[1,3,4]thiadiazol-2-yl]-urea;
1-(4-{3-[5-(3,5-Dichloro-2-hydroxy-phenyl)-[1,3,4]thiadiazol-2-yl]-ureido}-benzoyl)-1H-pyrrole-2-carboxylic acid ethyl ester;
4-{3-[5-(2-Hydroxy-5-nitro-phenyl)-[1,3,4]thiadiazol-2-yl]-ureido}-benzoic acid ethyl ester;
4-{3-[5-(2-Hydroxy-4-methoxy-phenyl)-[1,3,4]thiadiazol-2-yl]-ureido}-benzoic acid ethyl ester;
4-{3-[5-(2-Hydroxy-3-methoxy-phenyl)-[1,3,4]thiadiazol-2-yl]-ureido}-benzoic acid ethyl ester;
4-{3-[5-(2-Hydroxy-6-methoxy-phenyl)-[1,3,4]thiadiazol-2-yl]-ureido}-benzoic acid ethyl ester;
Ammonium, 2-{5-[3-(4-ethoxycarbonyl-phenyl)-ureido]-[1,3,4]thiadiazol-2-yl}-4,6-dinitro-phenylate;
4-{3-[5-(5-Chloro-2-hydroxy-3-methoxy-phenyl)-[1,3,4]thiadiazol-2-yl]-ureido}-benzoic acid ethyl ester;
1-(4-Benzoyl-phenyl)-3-[5-(3,5-difluoro-2-hydroxy-phenyl)-[1,3,4]thiadiazol-2-yl]-urea;
4-{3-[5-(3,4,5-Trifluoro-2-hydroxy-phenyl)-[1,3,4]thiadiazol-2-yl]-ureido}-benzoic acid ethyl ester;
1-(4-Benzoyl-phenyl)-3-[5-(2,3,5-trichloro-6-hydroxy-phenyl)-[1,3,4]thiadiazol-2-yl]-urea;
(ii) a pharmaceutically acceptable salt, prodrug, stereoisomer or solvate of (i) thereof.

### Methods of Preparation

The compounds of the present invention may be synthesized by many methods available to those skilled in the art of organic chemistry. General synthetic schemes for preparing compounds of the present invention are described below. These schemes are illustrative and are not meant to limit the possible techniques one skilled in the art may use to prepare the compounds disclosed herein. Further, the schemes presented herein are not intended to be limited to the particular depicted compounds of formula (I), e.g. intermediate and compounds in which A=aryl (including phenyl) or heteroaryl may be prepared by procedures depicted in the schemes. Different methods to prepare the compounds of the present invention will be evident to those skilled in the art. Additionally, the various steps in the synthesis may be performed in an alternate sequence in order to give the desired compound or compounds. Examples of compounds of the present invention prepared by methods described in the general schemes are given.

Scheme 1 illustrates the general synthesis of intermediates of formula (e). Starting materials described by formula (a) are commercially available or may be obtained, for example, by formylation of the corresponding phenyl by hexamethylene tetramine in trifluoroacetic acid. Intermediate (b) may be obtained by the treatment of (a) with thionyl chloride. The subsequent reaction of (b) with thiosemicarbazide gives a compound of formula (c). Cyclization of formula (c) compounds to give the thiadiazole intermediate (e) is achieved in refluxing toluene in the presence of methane sulfonic acic, or in refluxing PPA, or by other methods reported in literature. Reaction of the thiadiazole amine (d) with CDI gives intermediate (e).

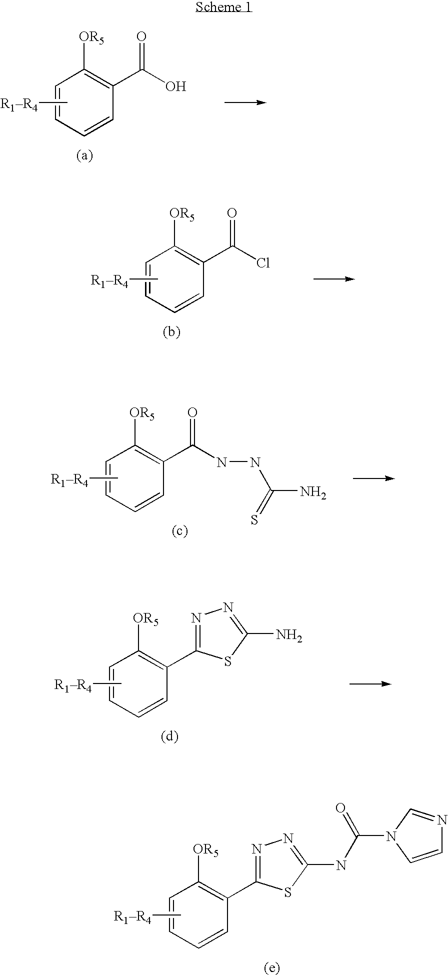

As illustrated in Scheme 2, compounds of formula (I), depicted as formula (f) in Scheme 2, can be obtained by reaction of the diathiazole amine(d) with isocyanates, in refluxing THF, dioxane or DMF; or by the reaction of the amine (d) with amines and one equivalent of triphosgene. Alternatively, compounds of formula (e) may be treated with one equivalent of aniline in refluxing THF and dioxane or DMF to give compounds of formula I, illustrated by compound (f) in Scheme 2.

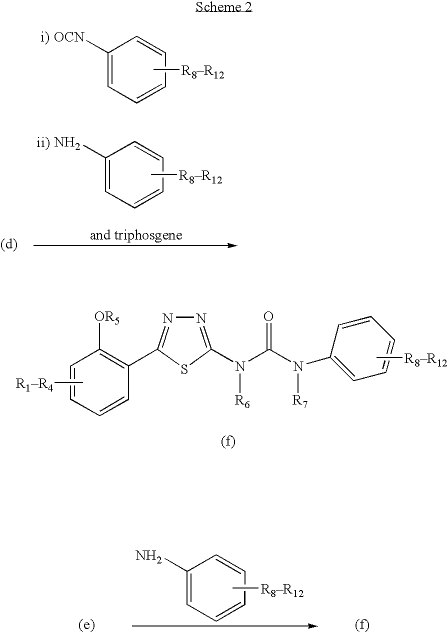

Further elaboration of the phenyl (or aryl or heteroaryl) urea may be accomplished by methods well-known in the art. For example, the carboxylic acid derivative (f) may be obtained by hydrolysis from the corresponding alkyl ester by under typical conditions, such as refluxing with KOH in methanol. Subsequently, as illustrated in Scheme 3, the resultant carboxylic acid (f) may be condensed with anilines in presence of typical condensing reagents such as EDCI, HOBt to obtain compounds of formula (g).

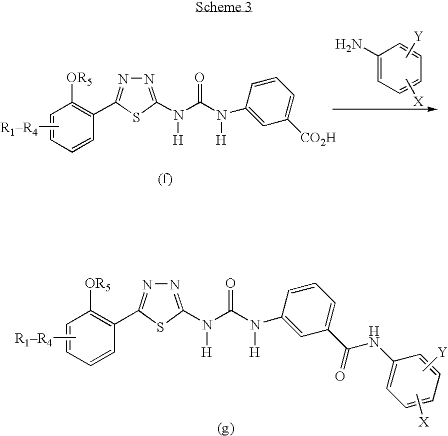

### Utility

The compounds of this invention are inhibitors of PAI-1 and are useful for the treatment or prevention of thromboembolic disorders in mammals (i.e., PAI-1 associated disorders). In general, a thromboembolic disorder is a circulatory disease caused by blood clots (i.e., diseases involving fibrin formation, platelet activation, and/or platelet aggregation). The term “thromboembolic disorders” as used herein includes arterial cardiovascular thromboembolic disorders, venous cardiovascular thromboembolic disorders, thromboembolic disorders in the chambers of the heart and fibrin accumulation in the microcirculation as occurs in disseminated intravascular coagulation. The term “thromboembolic disorders” as used herein also includes specific disorders selected from, but not limited to, unstable angina or other acute coronary syndromes, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis. It is noted that thrombosis includes occlusion (e.g., after a bypass) and reocclusion (e.g., during or after percutaneous transluminal coronary angioplasty). The thromboembolic disorders may result from conditions including but not limited to atherosclerosis, surgery or surgical complications, prolonged immobilization, arterial fibrillation, congenital thrombophilia, cancer, diabetes, effects of medications or hormones, and complications of pregnancy. The anti-thrombotic effect of compounds of the present invention is believed to be due to inhibition of PAI-1.

The effectiveness of compounds of the present invention as inhibitors of PAI-1, can be determined using a relevant purified serine protease, respectively, and an appropriate synthetic substrate. The rate of hydrolysis of the chromogenic or fluorogenic substrate by the relevant serine protease was measured both in the absence and presence of compounds of the present invention. Hydrolysis of the substrate resulted in the release of pNA (para nitroaniline), which was monitored spectrophotometrically by measuring the increase in absorbance at 405 nm. A decrease in the rate of absorbance or fluorescence change in the presence of inhibitor is indicative of enzyme inhibition. Such methods are known to one skilled in the art. The results of this assay are expressed as the inhibitory constant, Kᵢ.

Chromogenic assays for PAI-1 inhibitors were conducted using either tPA or Urokinase as a “substrate” for PAI-1 at a final assay volume of 200 uL with 1% DMSO, a final concentration of either 2 nM t-PA ((Alteplase, 1 mg/ml reconstituted from InTime 1 kit #193) or 10 units/mL urokinase (Abbokinase, Abbott, Chicago, Ill.). An amount of human PAI-1 (Molecular Innovations, Inc, PAI-A) sufficient to neutralize t-PA or urokinase was used. The chromogenic assay was started by adding either spectrozyme tPA substrate (American Diagnotica, Inc. CT, #444L) or S2444 from chromogenix as the substrate for urokinase. The inhibition of PAI-1 activity was determined by comparing the rate of the reactions in the absence of inhibitor, but in the presence of PAI-1. Compounds tested in the chromogenic assay are considered to be active if they exhibit Kᵢ's of equal to or less than 30 μM.

Human fibrinolysis assays were conducted by diluting the compounds of interest into assay buffer consisting of 50 mM potassium phosphate, pH 7.4 and 0.05% BSA. Human PAI-1 (Molecular Innovations Inc., Michigan, CAT #PAI-A), then tPA (Genetech, CA), human glu-plasminogen (Enzyme research laboratories (ERL), Indiana HPg2001), and plaminogen depleted human fibrinogen (ERL, HFN) were added sequentially, then followed by the addition of human thrombin (ERL, HT1002). Compounds tested in the fibrinolysis assay are considered to be active if they exhibit a Kᵢ of equal to or less than 30 μM.

Compounds of the present invention have demonstrated Kᵢ values of equal to or less than 30 μM, preferably less than 10 μM, more preferably less than 2 μM, and even more preferably less than 1.5 μM in at least one of the above assays, thereby confirming the utility of the compounds of the present invention as effective inhibitors of PAI-1 and useful for the prevention or treatment of thromboembolic disorders in mammals.

The antithrombotic effect of compounds of the present invention can be can be demonstrated using relevant in vivo thrombosis model includingin a rat model of acute venous thrombosis where the thrombolytic state is induced by submaximal tPA.

In this model male SD rats (300-400 g) are anesthetized with 60 mg/kg, i.p. Na-pentobarbital. PE-205 tubing is inserted in the trachea to maintain airway patency. PE-50 catheters are inserted in both jugular veins to administer test articles and in a femoral vein to inject human recombinant tissue factor (hrTF). The vena cava is isolated by a midline abdominal incision and temperature maintained with heating lamps. A fixed stenosis is produced just distal to the renal veins by tying a ligature around a 26-gauge steel tubing that was laid alongside the vena cava segment and then removing the tubing. Thrombosis is induced with a 1.4 mL/kg infusion of hrTF (1/10 dilution RecombiPlasTin®, Ortho Diagnostics) given over 2 min. The vena cava thrombus was removed and weighed 20 min after the start of hrTF infusion.

Treatment protocol includes i.v. infusion of a PAI-1 inhibitor of the present invention followed, after 5 minutes, by an i.v. infusion of human recombinant tissue plasminogen activator (tPA, Activase, Genentech) at a submaximal dose of 10 μg/kg/min. The hrTF is administered 1 min into the tPA infusion. The PAI-1 inhibitors are administered as a loading i.v. injection plus sustaining i.v. infusion at appropriate dose levels (mg/kg+mg/kg/hr) e.g.: at 3+3 and 10+10, and 2+2 and 5+5. Both tPA and PAI-1 inhibitor infusions are maintained until thrombus removal. The administration of the compounds of the present invention to rats treated with the submaximal dose of tPA results in significantly reduced thrombus weight.

The compounds of formula I and salts thereof are inhibitors of PAI-1, a major regulatory component of the plasminogen-plasmin system. As such, compounds of the present invention are useful in the treatment, inhibition, prevention or prophylaxis in a mammal, preferably in a human, of processes involving the production and/or action of PAI-1. See e.g. Binder et al., News Physiol. Sci., 17: 56-61 (2002) and Tsikouris, J. et al., J. Clin. Pharmacol., 42:1187-1199 (2002).

Accordingly, the compounds of the present invention may also be used in treating conditions including, but not limited to, metabolic diseases correlated to triacylglycerol levels & insulin resistance such as diabetes mellitus (Type 1 & 2), hyperinsulinemia, hyperglycemia and hypertriglyciridemia; obesity, acute & chronic inflammatory lung disorder such as respiratory distress syndrome, asthma, COPD, idiopathic pulmonary fibrosis, hyperoxide lung injury and bronchopulmonary dysplasia; renal disorders such as nephritic syndrome and hemolytic uremic syndrome; and malignancies such as tumor cell invasion, metastasis and neovascularization.

The compounds of the invention are also useful for the treatment of blood and blood products used in dialysis, blood storage in the fluid phase, especially ex vivo platelet aggregation. The present compounds may also be added to human plasma during the analysis of blood chemistry in hospital settings to determine the fibrinolytic capacity thereof.

The compounds of the present invention may also be used to treat cancer including, but not limited to, breast and ovarian cancer, and as imaging agents for the identification of metastatic cancers.

The compounds of the invention may also be used in the treatment of Alzheimer's disease. This method may also be characterized as the inhibition of plasminogen activator by PAI-1 in a mammal, particularly a human, experiencing or subject to Alzheimer's disease. This method may also be characterized as a method of increasing or normalizing levels of plasmin concentration in a mammal, particularly those experiencing or subject to Alzheimer's disease.

The compounds of the invention may be used for the treatment of myelofibrosis with myeloid metaplasia by regulating stromal cell hyperplasia and increases in extracellular matrix proteins.

The compounds of the invention may also be used in conjunction with protease inhibitor-containing highly active antiretroviral therapy (HAART) for the treatment of diseases which orginate from fibrinolytic impairment and hyper-coagulability of HIV-1 infected patients receiving such therapy.

The compounds of the invention may be used for the treatment of diabetic nephropathy and renal dialysis associated with nephropathy.

The compounds of the invention may be used to treat polycystic ovary syndrome, organ transplant rejection, septic shock and vascular damage associated with infections, cancer, septicemia, obesity, insulin resistance, proliferative diseases such as psoriasis, improving coagulation homeostasis, cerebrovascular diseases, microvascular disease, hypertension, dementia, arthritis, asthma, heart failure, arrhythmia, angina, and as a hormone replacement agent, treating, preventing or reversing progression of atherosclerosis, Alzheimer's disease, osteoporosis, osteopenia; reducing inflammatory markers, reducing C-reactive protein, or preventing or treating low grade vascular inflammation, stroke, coronary heart disease, primary and secondary prevention of myocardial infarction, stable and unstable angina, primary prevention of coronary events, secondary prevention of cardiovascular events, peripheral vascular disease, peripheral arterial disease including peripheral arterial occlusion, acute vascular syndromes, reducing the risk of undergoing a myocardial revascularization procedure, microvascular diseases such as nephropathy, neuropathy, retinopathy and nephrotic syndrome, hypertension, Type 1 and 2 diabetes and related diseases, hyperglycemia, hyperinsulinemia, malignant lesions, premalignant lesions, gastrointestinal malignancies, liposarcomas and epithelial tumors, proliferative diseases such as psoriasis, improving coagulation homeostasis, and/or improving endothelial function, and all forms of cerebrovascular diseases.

The compounds of the invention may be used for the topical applications in wound healing for prevention of scarring.

The compounds of the present invention can be administered alone or in combination with one or more additional therapeutic agents. These include anti-coagulant or coagulation inhibitory agents, anti-platelet or platelet inhibitory agents, thrombin inhibitors, or thrombolytic or fibrinolytic agents.

The compounds are administered to a mammal in a therapeutically effective amount. By “therapeutically effective amount” it is meant an amount of a compound of the present invention that, when administered alone or in combination with an additional therapeutic agent to a mammal, is effective to treat (i.e. prevent, inhibit or ameliorate) the thromboembolic disease condition or treat the progression of the disease in a host.

The compounds of the invention are preferably administered alone to a mammal in a therapeutically effective amount. However, the compounds of the invention can also be administered in combination with an additional therapeutic agent, as define below, to a mammal in a therapeutically effective amount. When administered in a combination, the combination of compounds is preferably, but not necessarily, a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased antiviral effect, or some other beneficial effect of the combination compared with the individual components.

By “administered in combination” or “combination therapy” it is meant that the compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

Compounds which can be administered in combination with the compounds of the present invention include, but are not limited to, anticoagulants, anti-thrombin agents, anti-platelet agents, fibrinolytics, hypolipidemic agents, antihypertensive agents, and anti-ischemic agents.

Anticoagulant agents (or coagulation inhibitory agents) that may be used in combination with the compounds of this invention include warfarin, heparin, low molecular weight heparin (for example LOVANOX™), as well as other factor VIIa, VIIIa, IXa, Xa, XIa, prothrombin, TAFI, and fibrinogen inhibitors known in the art. The term anti-platelet agents (or platelet inhibitory agents), as used herein, denotes agents that inhibit platelet function such as by inhibiting the aggregation, adhesion or granular secretion of platelets. Such agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, and piroxicam, including pharmaceutically acceptable salts, hydrates or prodrugs thereof. Of the NSAIDS, aspirin (acetylsalicylic acid or ASA), and piroxicam are preferred. Other suitable anti-platelet agents include clopidrogel and ticlopidine, including pharmaceutically acceptable salts, hydrates or prodrugs thereof. Ticlopidine is also a preferred compound since it is known to be gentle on the gastro-intestinal tract in use. Still other suitable platelet inhibitory agents include IIb/IIIa antagonists, thromboxane-A2-receptor antagonists and thromboxane-A2-synthetase inhibitors, prostacyclin mimetics, phosphodiesterase (PDE) inhibitors, such as dipyridamole or cilostazol, serotonin-2-receptor antagonists, and P2Y₁ and P2Y₁₂ receptor antagonists, as well as pharmaceutically acceptable salts, hydrates or prodrugs thereof. Preferred P2Y₁₂ receptor antagonists include ticlopidine and clopidogrel, including pharmaceutically acceptable salts, hydrates or prodrugs thereof. Clopidogrel is an even more preferred agent. Ticlopidine and clopidogrel are also preferred compounds since they are known to be gentle on the gastro-intestinal tract in use.

The term thrombolytics (or fibrinolytic) agents (or thrombolytics or fibrinolytics), as used herein, denotes agents that lyse blood clots (thrombi). Such agents include tissue plasminogen activator (TPA), anistreplase, urokinase, streptokinase, PAI-1 inhibitors, and inhibitors of alpha-2-antiplasmin, including pharmaceutically acceptable salts, hydrates or prodrugs thereof. The term anistreplase, as used herein, refers to anisoylated plasminogen streptokinase activator complex, as described, for example, in European Patent Application No. 028,489, the disclosure of which is hereby incorporated herein by reference herein. The term urokinase, as used herein, is intended to denote both dual and single chain urokinase, the latter also being referred to herein as prourokinase.

The term hypolipidemic agents, as used herein, includes HMG-CoA reductase inhibitors (for example, pravastatin, simvastatin, atorvastatin, and the like) and microsomal triglyceride transport protein inhibitors.

The term antihypertensive agents, as used herein, includes angiotensin-converting enzyme inhibitors (for example captopril, lisinopril, or fosinopril), angiotensin-II receptor antagonists (for example irbestatin, losartan, or valsartan), ACE/NEP inhibitors (for example omapatrilat or gemopatrilat), diuretics (for example furosemide, chlorothiazide, or amiloride) and β-blockers (for example propanolol, nadolo, or carvedilol).

Administration of the compounds of the present invention of the invention in combination with such additional therapeutic agent, may afford an efficacy advantage over the compounds and agents alone, and may do so while permitting the use of lower doses of each. A lower dosage minimizes the potential of side effects, thereby providing an increased margin of safety.

The compounds of the present invention are also useful as standard or reference compounds, for example as a quality standard or control, in tests or assays involving the inhibition of PAI-1. Such compounds may be provided in a commercial kit, for example, for use in pharmaceutical research involving PAI-1. For example, a compound of the present invention could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, compounds according to the present invention could be used to test their effectiveness.

The compounds of the present invention may also be used in diagnostic assays involving PAI-1 tissue-type plasminogen activator (“tPA”) and urinary type plasminogen activator (“uPA”). For example, the presence of tPA and/or uPA in an unknown sample could be determined by addition of the relevant chromogenic substrate, for example S2444 for uPA and spectrozyme (American Diagnostics, Inc., CT #444L) for tPA, to a series of solutions containing test sample and optionally one of the compounds of the present invention. If production of pNA is observed in the solutions containing test sample together with exogenous PAI-1 and a compound of the present invention, then one would conclude that selective PAI-1 activity was absent, rather than, for example, PAI-2 or PAI-3 activity.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt or hydrate form thereof; and (c) a package insert stating that the pharmaceutical composition can be used for the treatment of a thromboembolic disorder (as defined previously). In another embodiment, the package insert states that the pharmaceutical composition can be used in combination (as defined previously) with a second therapeutic agent to treat a thromboembolic disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

### Biological Assays

Chromogenic Assays for Inhibitors of PAI-1.

Chromogenic assays for PAI-1 inhibitors were conducted in 96 well plates (Costar 25381-054). Reactions were conducted using either tPA or Urokinase as a “substrate” for PAI-1. Reactions were set up such that each well contained a final volume of 200 uL with 1% DMSO, a final concentration of either 2 nM t-PA ((Alteplase, 1 mg/ml reconstituted from InTime 1 kit #193) or 10 units/mL urokinase (Abbokinase, Abbott, Chicago, Ill.). An amount of human PAI-1 (Molecular Innovations, Inc, PAI-A) sufficient to neutralize t-PA or urokinase was used. Each well contained 50 uL of buffer (50 mM Tris pH 8.3, 0.1 M NaCl containing 100 uL of Tween 80/L; buffer was filtered using a 0.2 um filter) and 2 uL of each compound of interest which had been diluted in 100% DMSO. Next, 50 uL of 10.4 nM PAI-1, in buffer, was added and the plate was vortexed for 1 min followed by a 30 minute incubation at room temperature. Fifty microliters of either 8 nM t-PA or 40 units/ml urokinase was added and the plate was vortexed for 1 minute. The chromogenic assay was started by adding either 50 uL of 1 mM spectrozyme tPA substrate (American Diagnotica, Inc. CT, #444L) or 50 uL of the 0.4 mM S2444 from chromogenix as the substrate for urokinase. The absorbance was measured at 405 nm over 15 mins using the kinetic mode of a spectramax 190 plate reader (Molecular Devices). The inhibition of PAI-1 activity was determined by comparing the rate of the reactions in the absence of inhibitor, but in the presence of PAI-1.

Human Fibrinolysis Assay Protocol

Human fibrinolysis assays were conducted in 96 well plates (Costar 25381-054). Stock solutions were diluted into assay buffer consisting of 50 mM potassium phosphate, pH 7.4 and 0.05% BSA. Compounds to be tested were serially diluted in 100% DMSO. Each well contained 50 uL assay buffer and 2 uL of a given concentration of the compound of interest. Fifty microliters of a 2 nM human PAI-1 (Molecular Innovations Inc., Michigan, CAT #PAI-A) was added to each well and the plate was incubated at room temperature for 5 minutes. Next, 50 uL of each of the following solutions was added to each well and mixed for one minute: 0.42 nM tPA (Genetech, CA), 800 nM human glu-plasminogen (Enzyme research laboratories (ERL), Indiana HPg2001), 2 mg/ml plaminogen depleted human fibrinogen (ERL, HFN). Finally, the reaction was initiated by the addition of 50 uL of 14 nM human thrombin (ERL, HT1002). The time course of the reaction was followed by measuring the absorbance at 405 nm using a spectramax plate reader in kinetic mode. The time course of the reaction was observed for 4 hours. The extended time course was used to monitor the shape of the reaction curve. The percent inhibition of the fibrinolysis was determined by comparing the absorbance values at the 4 hour time point for wells containing compounds and control wells which contained no compound.

### EXAMPLES

The following Examples illustrate embodiments of the inventive compounds and starting materials, and are not intended to limit the scope of the claims. For ease of reference, the following abbreviations are used herein:

Abbreviations

CH₃CN=acetonitrile
DCC=dicyclohexylcarbodiimide
DCE=dichloroethane
DCM=dichloromethane
DMAP=4-dimethylaminopyridine
DIPEA or DIEA=N,N-diisopropylethylamine
DME=1,2-dimethoxyethane
DMF=dimethyl formamide
EDCI=1-3-dimethylaminopropyl)-3-ethylcarbodiimide
Et₂O=diethyl ether
HOBT=1-hydroxybenzotriazole
EtOAc=ethyl acetate
HCl=hydrochloric acid
KOH=potassium hydroxide
K₂CO₃=potassium carbonate
LiAlH₄=lithium aluminum hydride
MeCN=acetonitrile
MeOH=methanol
MgSO₄=magnesium sulfate
NaH=sodium hydride
NaOH=sodium hydroxide
NMP=1-methyl-2-pyrrolidinone
SOCl₂=thionyl chloride
TEA=triethylamine
bp=boiling point
g=gram(s)
mg=milligram(s)
ml=milliliter
μl=microliter
l=liter
mmol=millimole
[mol=micromole
mol=mole
mp=melting point
RT=room temperature
Rt=retention time
NMR (Nuclear Magnetic resonnance was performed on a Brucker 200 spectrometer
(s=singulet, d=doublet, t=triplet, dd=doublet of doublet, q=quadruplet, m=multiplet)
Elementary analysis were carried on a Carlo-Erba Mod 106 elementary analyzer

### Preparation 1

### 3,5-Dichloro-2-hydroxy-benzoyl Chloride

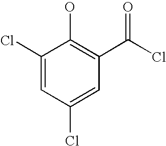 25 g of 3,5-dichlorosalicylic acid were suspended in 150 ml dichloromethane and 30 ml of thionyl chloride and 0.5 ml of DMF were added. The mixture was refluxed until a clear solution was obtained and the solvent evaporated, the temperature being maintained below 40° C., to yield a product used as such in Preparation 2.

¹H NMR (DMSO-d₆): 9.92 (1H,s), 7.98 (1H, d, J=2 Hz), 7.68 (1H, d, J=2 Hz)

### Preparation 2

### 2-(5-Amino-[1,3,4]thiadiazol-2-yl)-4,6-dichloro-phenyl

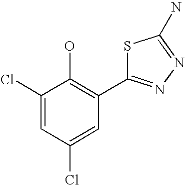

The product of Preparation 1 was dissolved in 100 ml THF, and 35 g of thiosemicarbazide dissolved in 120 ml DMF were added drop by drop. After 2 hours, the solvent was evaporated, the residue washed by 10% HCl water, filtered and dissolved in 250 ml toluene. 17 ml of methane sulfonic acid were added and the mixture was refluxed for 4 hours. After return to RT, the obtained precipitate was filtered, washed with acetone and with ammonia. The obtained precipitate was washed by hot acetone to give 16 g of a product melting at 268° C.

¹H NMR (DMSO-d₆): 7.78 (1H, d, J=2 Hz), 7.75 (2H, s), 7.58 (1H, d, J=2 Hz)

Preparation 3

### Imidazole-1-carboxylic Acid [5-(3,5-dichloro-2-hydroxy-phenyl)-[1,3,4]thiadiazol-2-yl]-amide

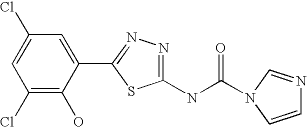 4.6 g of the product of Preparation 3 were suspended in 100 ml THF and 3.6 g of CDI were added. The mixture was stirred for 1 night at RT, the precipitate filtered and washed with acetone to give 4.8 g of a white solid.

¹H NMR (DMSO-d₆): 8.36 (1H, s), 7.75 (1H, s), 7.68 (1H, d, J=2 Hz), 7.65(1H, d, J=2 Hz), 7.03 (1H, s)

### Example 1

### 1-[5-(3,5-Dichloro-2-hydroxy-phenyl)-[1,3,4]thiadiazol-2-yl]-3-(4-nitro-phenyl)-urea

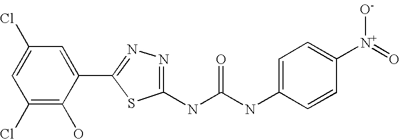 520 mg of the product of Preparation 2 were dissolved in THF and 400 mg of 4-nitrophenylisocyanate were added. The mixture was refluxed for 3 hours, the solvent was evaporated and washed wit hot acetone to give 670 mg of a product melting over 350° C.

Microanalysis: theory (%): C: 42.3; H: 2.13; N: 16.4; obtained (%): C: 42.0, H: 2.0; N: 16.1

### Example 2

### 4-{3-[5-(3,5-Dichloro-2-hydroxy-phenyl)-[1,3,4]thiadiazol-2-yl]-ureido}-benzoic Acid Ethyl Ester

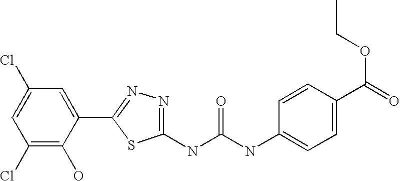 3 g of the product of Preparation 2 were dissolved in xylene, 2.4 g of ethyl-4-isocyanatobenzoate and one drop of pyridine were added, and the mixture was refluxed for 3 hours. A precipitate was formed, filtered from hot xylene and washed with acetone to give 4.6 g of an off-white product of melting point >300° C.

Microanalysis: theory (%): C: 47.7; H: 3.11; N: 12.4; obtained (%): C: 48.0, H: 3.25; N: 12.2

### Example 3 and 4

The products of Example 3 and 4 were obtained starting from the product of Preparation 2 and commercially available isocyanates.

### Example 3

### 3-{3-[5-(3,5-Dichloro-2-hydroxy-phenyl)-[1,3,4]thiadiazol-2-yl]-ureido}-benzoic Acid Methyl Ester

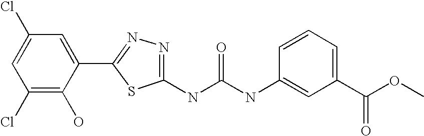

Mp: 305° C.

Microanalysis: theory (%): C: 46.5; H: 2.75; N: 12.8; obtained (%): C: 46.5, H: 2.72; N: 12.7

### Example 4

### 4-{3-[5-(3,5-Dichloro-2-hydroxy-phenyl)-[1,3,4]thiadiazol-2-yl]-ureido}-benzoic Acid Methyl Ester

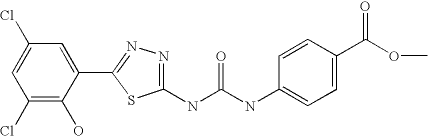

Mp: 330° C.

Microanalysis: theory (%): C: 46.5; H: 2.75; N: 12.8; obtained (%): C: 46.8, H: 2.87; N: 12.6

### Example 5

### 3-{3-[5-(3,5-Dichloro-2-hydroxy-phenyl)-[1,3,4]thiadiazol-2-yl]-ureido}-benzoic Acid

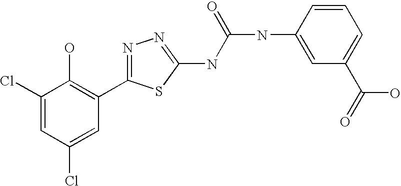 To a solution of 100 mg of the product of Example 3 in 10 ml methanol was added 0.1 ml of concentrated KOH and water and the mixture was refluxed for 3 hours. The obtained solution was poured in water, acidified by concentrated HCl to give 60 mg of a product melting over 360° C.

Microanalysis: theory (%, 1.5H₂O): C: 42.5; H: 2.9; N: 12.4; obtained (%): C: 42.5, H: 2.7; N: 12.2

### Example 6

### 3-{3-[5-(3,5-Dichloro-2-hydroxy-phenyl)-[1,3,4]thiadiazol-2-yl]-ureido}-N-phenyl-benzamide

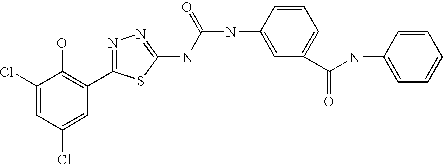

To a solution of 200 mg of the product of Example 5 in DMF was added 0.076 g of HOBt, 0.043 ml aniline, 0.10 ml of DIPEA and 0.11 g of EDCI. The reaction mixture was stirred for one night, poured in water and the resulting precipitate was crystallized in ethanol to give a product melting at 298° C.

Microanalysis: theory (%): C: 52.8; H: 3.02; N: 14.0; obtained (%): C: 52.7, H: 3.12; N: 14.3

### Example 7

### 3-{3-[5-(3,5-Dichloro-2-hydroxy-phenyl)-[1,3,4]thiadiazol-2-yl]-ureido}-N-(4-dimethylamino-phenyl)-benzamide

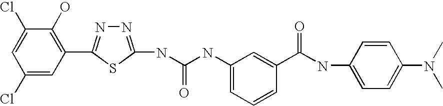

To a mixture of the product of Example 5 (30 mg, 0.071 mmole), 1-hydroxybenzotriazole HOBT (11.4 mg, 0.085 mmole) and p-dimethylaminoaniline (0.141 mmole) in DMF (1 ml) was added a solution of EDCI (0.085 mmole) and DIEA (0.085 mmole) in DMF (1 ml). The reaction mixture was stirred for 20 hours at 50° C. then allowed to cool down to RT and loaded on a SAX cartridge (conditioned with methanol). The cartridge was washed with methanol (3 mL), and the filtrate was collected. Water was added into it until a precipitate formed. The precipitate was filtered, washed with water then redissolved in THF. The organic solvent was evaporated under reduced pressure to give the desired compound. The final product was characterized by LC Mass (LCMS conditions: LC Micromass platform (APCI+, DAD (210-400 nm)), Column: TSK gel Super ODS 4.6 mm ID×5 cm, Flow rate: 2.75 mL/min, Gradient: from 100% eluent A to 100% eluent B in 2 min., with a plateau with 100% eluent B during 1 min. Eluent A: H₂O (0.05% TFA), Eluent B: CH₃CN/H₂O/TFA (80/20/0.05)).

LCMS: Rt: 2.01 nm; M/z: 543; Purity: 91%

### Example 8 to 14

The products of Examples 8 to 14 were obtained according to the procedure reported for Example 7 from the product of Example 5 and commercially available anilines.

### Example 8

### 3-{3-[5-(3,5-Dichloro-2-hydroxy-phenyl)-[1,3,4]thiadiazol-2-yl]-ureido}-N-(3-hydroxymethyl-phenyl)-benzamide

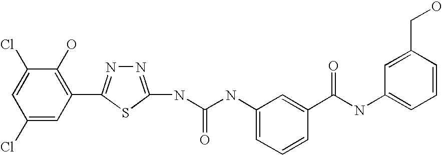

LCMS: Rt: 2.17 nm; M/z: 530; Purity: 100%

### Example 9

### 3-{3-[5-(3,5-Dichloro-2-hydroxy-phenyl)-[1,3,4]thiadiazol-2-yl]-ureido}-N-(2-methoxy-phenyl)-benzamide

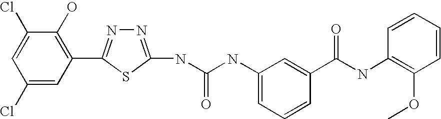

LCMS: Rt: 2.43 nm; M/z: 530; Purity: 91%

### Example 10

### 3-{3-[5-(3,5-Dichloro-2-hydroxy-phenyl)-[1,3,4]thiadiazol-2-yl]-ureido}-N-(4-methoxy-phenyl)-benzamide

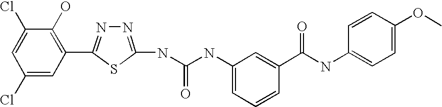

LCMS: Rt: 2.33 nm; M/z: 530; Purity: 100%

### Example 11

### 3-{3-[5-(3,5-Dichloro-2-hydroxy-phenyl)-[1,3,4]thiadiazol-2-yl]-ureido}-N-m-tolyl-benzamide

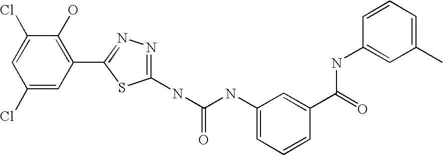

LCMS: Rt: 2.43 nm; M/z: 514; Purity: 97%

### Example 12

### 3-{3-[5-(3,5-Dichloro-2-hydroxy-phenyl)-[1,3,4]thiadiazol-2-yl]-ureido}-N-(4-pentyloxy-phenyl)-benzamide

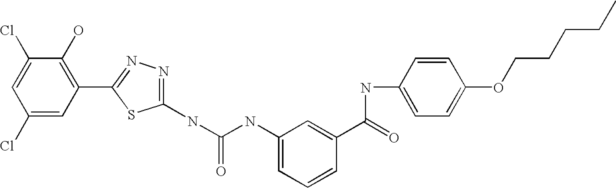

LCMS: Rt: 2.65 nm; M/z: 586 Purity: 93%

### Example 13

### 3-{3-[5-(3,5-Dichloro-2-hydroxy-phenyl)-[1,3,4]thiadiazol-2-yl]-ureido}-N-(1H-indol-5-yl)-benzamide

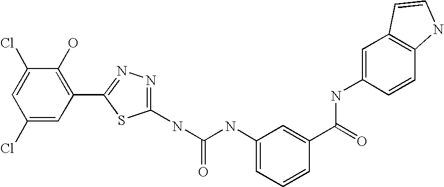

LCMS: Rt: 2.33 mn; M/z: 530; Purity: 100%

### Example 14

### 3-{3-[5-(3,5-Dichloro-2-hydroxy-phenyl)-[1,3,4]thiadiazol-2-yl]-ureido}-N-(4-phenoxy-phenyl)-benzamide

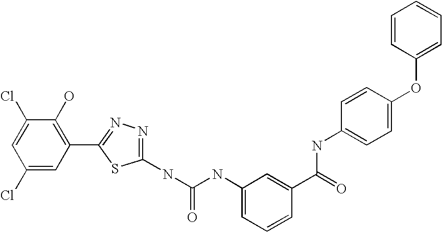

LCMS: Rt: 2.56 mn; M/z: 592; Purity: 87%

### Example 15

### 1-[5-(3,5-Dichloro-2-hydroxy-phenyl)-[1,3,4]thiadiazol-2-yl]-3-(3-oxo-1,3-dihydro-isobenzofuran-5-yl)-urea

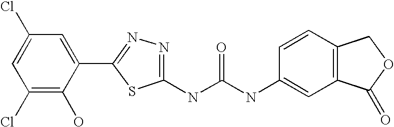

A mixture of the product of Preparation 3 (20 mg; 0.045 mmole and 6 aminophthalide (0.01 mmole) in DMF (1 ml) was stirred at 50° C. during 20 hours. Purification was done as followed: THF (500 □l) and HCl in isopropanol (5.5N; 100 □l) was added to the reaction mixture which then was loaded on a Strong Cation eXchange cartridge (SCX 150 mg conditioned with methanol then DMF). The organic solvent was evaporated under reduced pressure to give the desired compound. The final product was characterized by LC Mass (LCMS conditions: LC Micromass platform (APCI+, DAD (210-400 nm)), Column: TSK gel Super ODS 4.6 mm ID×5 cm, Flow rate: 2.75 mL/min, Gradient: from 100% eluent A to 100% eluent B in 2 min., with a plateau with 100% eluent B during 1 min. Eluent A: H₂O (0.05% TFA), Eluent B: CH₃CN/H₂O/TFA (80/20/0.05)).

LCMS: Rt: 2.24 mn; M/z: 437; Purity: 96%

### Example 16 to 28

The products of Example 16 to 28 were prepared according to the procedure of Example 15 using the product of Preparation 3 and commercially available anilines.

### Example 16

### 1-(2-Benzoyl-4-nitro-phenyl)-3-[5-(3,5-dichloro-2-hydroxy-phenyl)-[1,3,4]thiadiazol-2-yl]-urea

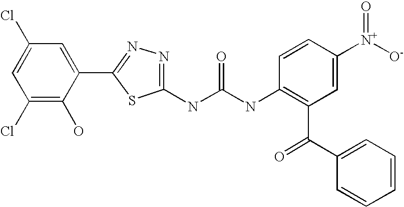

LCMS: Rt: 2.0 mn; M/z: 531; Purity: 97%

### Example 17

### 1-[5-(3,5-Dichloro-2-hydroxy-phenyl)-[1,3,4]thiadiazol-2-yl]-3-(4-hydroxy-phenyl)-urea

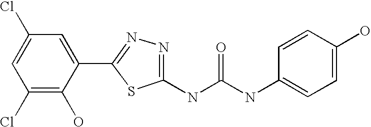

LCMS: Rt: 2.18 mn; M/z: 398; Purity: 86%

### Example 18

### 1-(4-Benzotriazol-2-yl-phenyl)-3-[5-(3,5-dichloro-2-hydroxy-phenyl)-[1,3,4]thiadiazol-2-yl]-urea

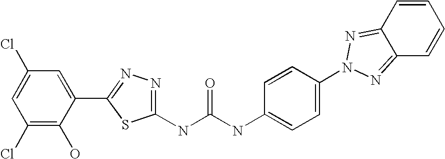

LCMS: Rt: 2.75 mn; M/z: 499; Purity: 98%

### Example 19

### 1-[4-(4-Bromo-1-methyl-1H-pyrazol-3-yl)-phenyl]-3-[5-(3,5-dichloro-2-hydroxy-phenyl)-[1,3,4]thiadiazol-2-yl]-urea

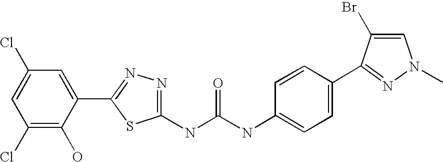

LCMS: Rt: 2.52 mn; M/z: 541; Purity: 96%

### Example 20

### 1-[5-(3,5-Dichloro-2-hydroxy-phenyl)-[1,3,4]thiadiazol-2-yl]-3-(4-imidazol-1-yl-phenyl)-urea

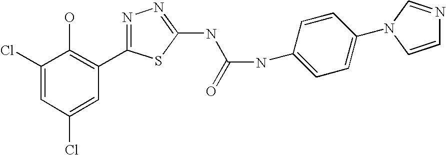

LCMS: Rt: 1.93 mn; M/z: 447; Purity: 97%

### Example 21

### 5-(4-{3-[5-(3,5-Dichloro-2-hydroxy-phenyl)-[1,3,4]thiadiazol-2-yl]-ureido}-phenyl)-4-methyl-furan-3-carboxylic Acid Ethyl Ester

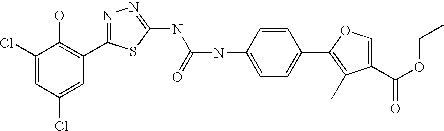

LCMS: Rt: 2.82 mn; M/z: 534; Purity: 100%

### Example 22

### 1-[5-(3,5-Dichloro-2-hydroxy-phenyl)-[1,3,4]thiadiazol-2-yl]-3-(4-[1,2,4]triazol-1-yl-phenyl)-urea

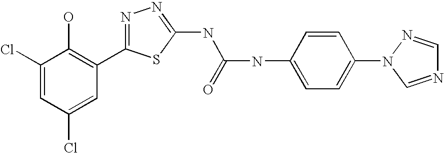

LCMS: Rt: 2.20 mn; M/z: 449; Purity: 100%

### Example 23

### 1-[5-(3,5-Dichloro-2-hydroxy-phenyl)-[1,3,4]thiadiazol-2-yl]-3-(9-oxo-9H-fluoren-3-yl)-urea

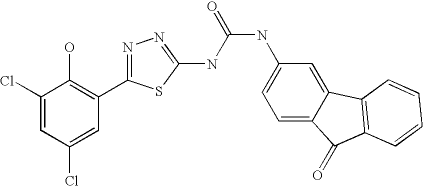

LCMS: Rt: 2.97 mn; M/z: 484; Purity: 95%

### Example 24

### 1-[5-(3,5-Dichloro-2-hydroxy-phenyl)-[1,3,4]thiadiazol-2-yl]-3-[4-(4-methoxy-benzoyl)-phenyl]-urea

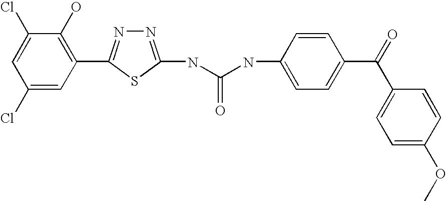

LCMS: Rt: 2.48 mn; M/z: 515; Purity: 100%

### Example 25

### 1-[5-(3,5-Dichloro-2-hydroxy-phenyl)-[1,3,4]thiadiazol-2-yl]-3-(1-oxo-1,3-dihydro-isobenzofuran-5-yl)-urea

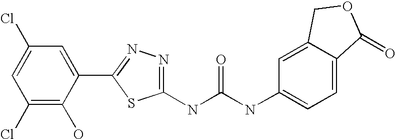

LCMS: Rt: 2.48 mn; M/z: 437: Purity: 100%

### Example 26

### 1-[5-(3.5-Dichloro-2-hydroxy-phenyl)-[1,3,4]thiadiazol-2-yl]-3-(2-methoxy-5-nitro-phenyl)-urea

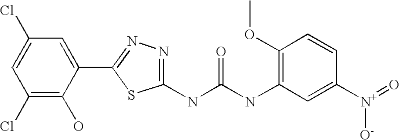

LCMS: Rt: 2.42 mn; M/z: 457; Purity: 100%

### Example 27

### 1-[5-(3,5-Dichloro-2-hydroxy-phenyl)-[1.3.4]thiadiazol-2-yl]-3-(2-fluoro-5-nitro-phenyl)-urea

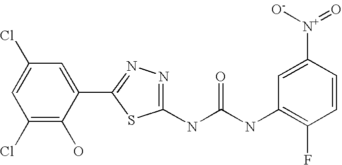

LCMS: Rt: 2.43 mn; M/z: 445; Purity: 100%

### Example 28

### 4-{3-[5-(3,5-Dichloro-2-hydroxy-phenyl)-[1.3.4]thiadiazol-2-yl]-ureido}-3-methyl-benzoic Acid Ethyl Ester

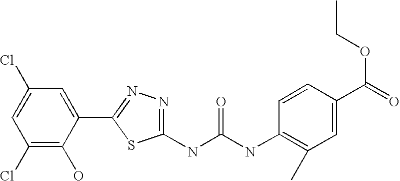

LCMS: Rt: 2.38 mn; M/z: 467; Purity: 99%

### Example 29

### 1-(4-Benzoyl-phenyl)-3-[5-(3,5-dichloro-2-hydroxy-phenyl)-[1,3,4]thiadiazol-2-yl]-urea

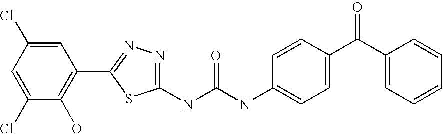 500 mg of the product of Preparation 3 and 200 mg of 4-aminobenzophenone were dissolved in THF and the mixture was refluxed for 8 hours. The hot solution was filtered and the precipitate was discarded. The filtrate was left to return to RT, and a precipitate was formed, which was washed with dilute HCl and acetone to give 80 mg of a yellow solid.

LCMS: Rt: 3.12 mn; M/z: 486; Purity: 96%

### Example 30

### 1-[5-(3,5-Dichloro-2-hydroxy-phenyl)-[1,3,4]thiadiazol-2-yl]-3-[4-(pyridine-4-carbonyl)-phenyl]-urea

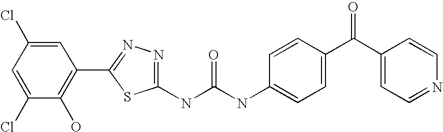 Intermediate 30-1

### N-[4-(Pyridine-4-carbonyl)-phenyl]-acetamide

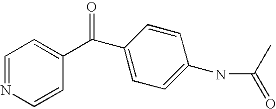 To 90 g of aluminium trichloride were added drop by drop 18 ml of DMF. 25 g of isonicotinoyl chloride and then 25 g of acetanilide were added portionwise to the mixture. The reaction mixture was heated to 90° C. during 2 hours. After return to RT, dichloromethane was added and the obtained solution was poured on ice/water. The dichloromethane solution was evaporated and the residue crystallized in isopropyl ether to give 900 mg of a product melting at 215° C.
Intermediate 30-2

### (4-Amino-phenyl)-pyridin-4-yl-methanone

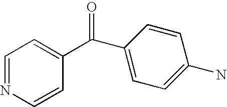 900 mg of intermediate 30-1 were dissolved in ethanol, 1 g of NaOH was added and the mixture was refluxed for 1 hour. Ethanol was then evaporated and the residue taken in water and filtered to give 670 mg of a product melting at 152° C.

670 mg of intermediate 30-2 and 825 mg of the product of Preparation 3 were dissolved in THF and the mixture was refluxed for 7 hours. THF was then evaporated and the residue crystallized in acetone and washed with methanol to give 300 mg of the product of Example 30, melting over 320° C.

¹H NMR (DMSO-d₆): 9.75 (1H,s), 8.82 (2H,d), 8.05 (1H, d), 7.86 (2H, d), 7.84 (2H,d), (7.68, 1H, d), 7.55 (d,2H)

### Example 31

### Hydrochloride of 1-[5-(3,5-Dichloro-2-hydroxy-phenyl)-[1,3,4]thiadiazol-2-yl]-3-[4-(pyridine-3-carbonyl)-phenyl]-urea

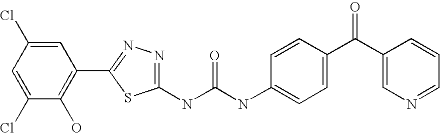 Intermediate 31-1

### N-[4-(Pyridine-3-carbonyl)-phenyl]-acetamide

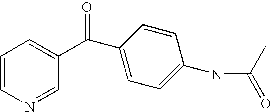

To 90 g of aluminium trichloride were added drop by drop 18 ml of DMF. 25 g of nicotinoyl chloride and then 25 g of acetanilide were added portionwise to the mixture. The reaction mixture was heated to 120° C. during 2 hours. After return to RT dichloromethane was added and the obtained solution was poured on ice/water. Sodium hydroxide was added and the dichloromethane phase was evaporated and the residue crystallized in methanol to give 5.5 g of a product melting at 215° C.

Intermediate 31-2

### (4-Amino-phenyl)-pyridin-3-yl-methanone

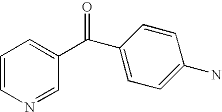

5.5 g of intermediate 31-1 were dissolved in ethanol, 10 g of NaOH was added and the mixture was refluxed for 2 hours. Ethanol was then evaporated and the residue taken in water, filtered and crystallized in isopropyl ether to give 1.5 g of a product melting at 107° C.

400 mg of intermediate 31-2 and 825 mg of the product of Preparation 3 were dissolved in THF and the mixture was refluxed for 8 hours. The hot THF solution was filtered and the obtained crystals were dissolved in DMF, a solution of HCl in isopropyl ether was added and the obtained precipitate was washed with methanol to give 120 mg of the product of Example 31, melting over 320° C.

¹H NMR (DMSO-d₆): 10.35 (1H,s), 9.05 (1H,s), 8.95 (1H,d), 8.45 (1H,d) 8.12 (1H,d), 8.09 (1H, d), 7.97 (1H, d), 7.89 (1H, d), 7.83 (2H, d), 7.76 (1H, d), 7.70 (2H, d)

### Example 32

### 1-[5-(3,5-Dichloro-2-hydroxy-phenyl)-[1,34]thiadiazol-2-yl]-3-[4-(4-methyl-benzoyl)-phenyl]-urea

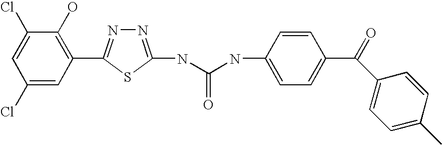 400 mg of 4-Amino-4′-methyl benzophenone (prepared according to Carmelino, Pestic. Sci., 1995 p. 227-236) and 500 mg of the product of Preparation 3 were dissolved in THF and the mixture was heated for 3 hours. After return to RT, a precipitate was filtered, washed by dilute HCl and acetone to give 220 mg of a product melting over 320° C.

Microanalysis: theory (%): C: 55.3; H: 3.23; N: 11.2; obtained (%): C: 55.3, H: 3.26; N: 11.2

### Example 33

### 1-[5-(3,5-Dichloro-2-hydroxy-phenyl)-[1,3,4]thiadiazol-2-yl]-3-[4-(4-fluoro-benzoyl)-phenyl]-urea

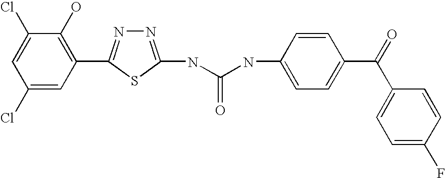

300 mg of 4-Amino-4′-fluoro benzophenone (prepared according to Carmelino, Pestic. Sci., 1995 p. 227-236) and 250 mg of the product of Preparation 3 were dissolved in THF and the mixture was heated for 6 hours. The hot solution was filtered and the obtained precipitate was washed by dilute HCl and methanol to give 100 mg of a product melting over 320° C.

Microanalysis: theory (%): C: 52.5; H: 2.60; N: 11.1; obtained (%): C: 52.3, H: 2.54; N: 11.4

### Example 34

### 1-[5-(3,5-Dichloro-2-hydroxy-phenyl)-[1,3,4]thiadiazol-2-yl]-3-[4-(2-fluoro-benzoyl)-phenyl]-urea

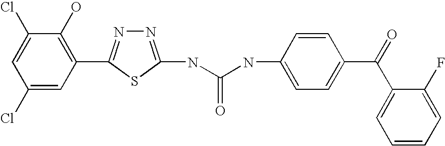 250 mg of 4-Amino-2′-fluoro benzophenone (prepared according to Carmelino, Pestic. Sci., 1995 p. 227-236) and 300 mg of the product of Preparation 3 were dissolved in dioxane and the mixture was heated for 10 hours. The hot solution was filtered and the obtained precipitate was washed by dilute HCl and methanol to give 250 mg of a product melting over 320° C.

Microanalysis: theory (%): C: 52.5; H: 2.60; N: 11.1; obtained (%): C: 52.1, H: 2.30; N: 11.4

### Example 35

### 1-[5-(3,5-Dichloro-2-hydroxy-phenyl)-[1,3,4]thiadiazol-2-yl]-3-(9-oxo-9,10-dihydro-acridin-3-yl)-urea

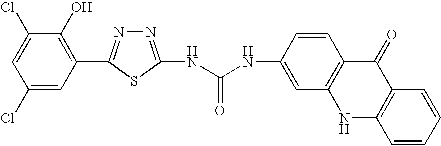 420 mg of 3-amino-10H-acridin-9-one (prepared according to Mannani, Eur. J. Med. Chem., 1991 p. 117-119) and 400 mg of the product of Preparation 3 were dissolved in dioxane and the mixture was heated for 12 hours. The hot solution was filtered and the obtained precipitate was washed by dilute HCl and methanol to give 86 mg of a product melting over 320° C.

Microanalysis: theory (%): C: 53.0; H: 2.53; N: 14.0; obtained (%): C: 52.8, H: 2.75; N: 13.8

### Example 36

### 1-[5-(3,5-Dichloro-2-hydroxy-phenyl)-[1,3,4]thiadiazol-2-yl]-3-(10-methyl-9-oxo-9,10-dihydro-acridin-3-yl)-urea

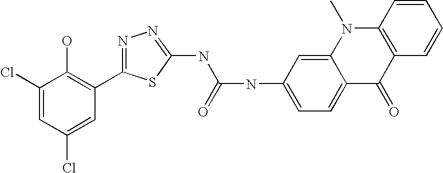

220 mg of 3-amino-10-Methyl-10H-acridin-9-one (prepared according to Cain, J. Med. Chem., 1976 p. 772-774) and 220 mg of the product of Preparation 3 were dissolved in dioxane and the mixture was heated for 12 hours. The hot solution was filtered and the obtained precipitate was washed by dilute HCl and methanol to give 38 mg of a product melting over 320° C.

Microanalysis: theory (%): C: 53.9; H: 2.95; N: 13.7; obtained (%): C: 53.6, H: 3.11; N: 13.4

### Example 37

### 1-[5-(3,5-Dichloro-2-hydroxy-phenyl)-[1,3,4]thiadiazol-2-yl]-3-(5-oxo-5,10-dihydro-benzo[b][1,8]naphthyridin-8-yl)-urea

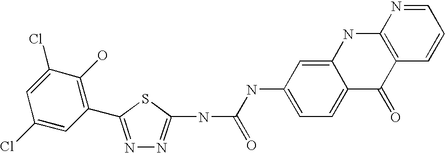 Intermediate 37-1

### 2-(3-Amino-phenylamino)-nicotinic acid methyl ester

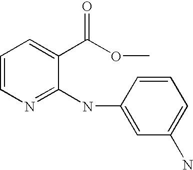 10 g of 2-(2-Nitro-phenylamino)-nicotinic acid methyl ester (obtained as described in Eur. J. Med. Chem. 1994, p. 441-446) was dissolved in methanol, Raney nickel was added and the mixture was submitted to hydrogenation by hydrogen at round temperature. When hydrogen absorption stopped, the reaction mixture was filtered, the methanol was evaporated and the residue crystallized in isopropyl ether to give 8.5 g. of a product used without further purification
Intermediate 37-2

### 2-(3-Amino-phenylamino)-nicotinic Acid

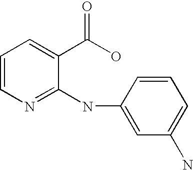 [0351] Intermediate 37-1 (8.5 g) was dissolved in ethanol and 15 ml of 10N sodium hydroxide were added. The mixture was refluxed for 2 hours and the solvent was evaporated. The residue was dissolved in water and acetic acid was added. 7 g of a white precipitate were obtained, which were used without further purification.
   Intermediate 37-3

### 8-Amino-10H-benzo[b][1,8]naphthyridin-5-one

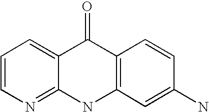 Intermediate 37-2 (2 g) was dissolved in 10 cc concentrated sulfuric acid and the mixture was heated at 160° C. for 6 hours. After return to RT, a solution of ammonia was added and the precipitate was filtered to give 350 mg of the expected product.

LCMS: Rt: 2.12 mn; M/z: 212; Purity: 94%

350 mg of intermediate 37-3 and 350 mg of the product of Preparation 3 were dissolved in dioxane and the mixture was heated for 7 hours. The hot solution was filtered and the obtained precipitate was washed by dilute HCl and methanol to give 86 mg of a product melting over 320° C.

LCMS: Rt: 3.25 mn; M/z: 500; Purity: 80%

### Example 38

### 1-[5-(3,5-Dichloro-2-hydroxy-phenyl)-[1,3,4]thiadiazol-2-yl]-3-[4-(thiophene-2-carbonyl)-phenyl]-urea

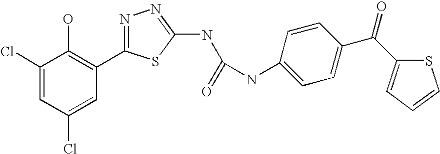 Intermediate 38-1

### (4-Amino-phenyl)-thiophen-2-yl-methanone

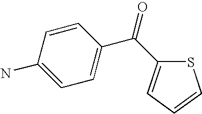 360 mg of (4-Nitro-phenyl)-thiophen-2-yl-methanone (prepared according to J. Chem. Soc. Perkin Trans 2, 1978, p. 1232-1234) were dissolved in ethanol and 1.6 g of tin dichloride was added. The mixture was refluxed for 3 hours. The solvent was evaporated and water and concentrated NaOH solution were added. After extraction with dichloromethane and evaporation, 170 mg of a white solid were obtained, which were used without further purification.

150 mg of intermediate 38-1 and 200 mg of the product of Preparation 3 were dissolved in dioxane and the mixture was heated for 8 hours. The hot solution was filtered and the obtained precipitate was washed by dilute HCl and methanol to give 102 mg of a product melting over 320° C.

LCMS: Rt: 2.87 mn; M/z: 491; Purity: 96%

### Example 39

### (3-{3-[5-(3,5-Dichloro-2-hydroxy-phenyl)-[1,3,4]thiadiazol-2-yl]-ureido}-9-oxo-9H-acridin-10-yl)-acetic Acid Ethyl Ester

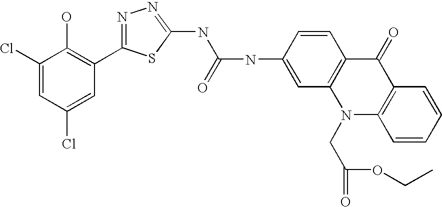 Intermediate 39-1

### (3-Nitro-9-oxo-9H-acridin-10-yl)-acetic Acid Ethyl Ester

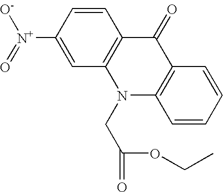 1.6 g of 3-nitro-10H-acridin-9-one (prepared according to Arzneim. Forsh, 2000, vol 50, no 2 p. 163-166) were dissolved in THF and one equivalent of NaH was added. The mixture was left at RT during 2 hours, and one equivalent of ethyl bromoacetate was added. The reaction mixture was then stirred for two hours at 70° C., and, after return to RT, water was added. The obtained precipitate was washed with acetone, taken in hot THF and a precipitate was discarded. THF was evaporated and the residue crystallized in isopropyl ether to give 0.73 g of intermediate 39-1
Intermediate 39-2

### (3-Amino-9-oxo-9H-acridin-10-yl)-acetic Acid Ethyl Ester

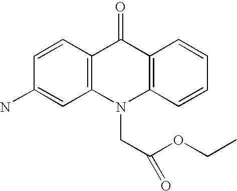 730 mg of intermediate 39-1 were dissolved in ethanol and 3.5 g of tin dichloride was added. The mixture was refluxed for 4 hours. The solvent was evaporated and water and concentrated ammonia solution were added. After extraction with dichloromethane and evaporation, 300 mg of a white solid were obtained, which were used without further purification. 300 mg of intermediate 39-2 and 350 mg of the product of Preparation 3 were dissolved in dioxane and the mixture was heated for 3 hours. The hot solution was filtered and the obtained precipitate was washed by acetone, dilute HCl and methanol to give 45 mg of a product melting over 320° C.

Microanalysis: theory (%): C: 53.4; H: 3.28; N: 12.0; obtained (%): C: 53.0, H: 3.33; N: 11.9

### Example 40

### 1-[5-(3,5-Dichloro-2-hydroxy-phenyl)-[1,3,4]thiadiazol-2-yl]-3-[4-(1H-pyrrole-2-carbonyl)-phenyl]-urea

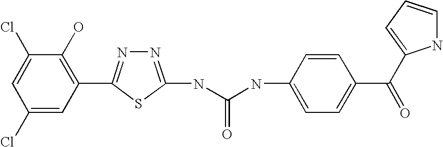 Intermediate 40-1

### (4-Amino-phenyl)-(1H-pyrrol-2-yl)-methanone

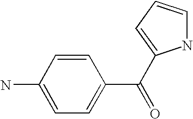

To 1.3 g of (4-nitro-phenyl)-(1H-pyrrol-2-yl)-methanone (prepared as in Chem Pharm Bull, 1997, vol. 11 p. 1767-1776) dissolved in ethanol was added 5.6 g of tin chloride. The mixture was refluxed for 4 hours. The solvent was evaporated and water and concentrated NaOH solution were added. After extraction with dichloromethane and evaporation, 1 g of a white solid were obtained, which were used without further purification.

1 g of intermediate 40-1 and 400 mg of the product of Preparation 3 were dissolved in dioxane and the mixture was refluxed for 3 hours. The hot solution was filtered and the obtained precipitate was washed by acetone, dilute HCl and methanol to give 74 mg of a product melting over 320° C.

Microanalysis: theory (%, 1H₂O): C: 48.8; H: 3.07, N: 14.2; obtained (%): C: 49.1, H: 2.96; N: 13.9

### Example 41

### 1-[5-(3,5-Dichloro-2-hydroxy-phenyl)-[1,3,4]thiadiazol-2-yl]-3-[4-(1-methyl-1H-pyrrole-2-carbonyl)-phenyl]-urea

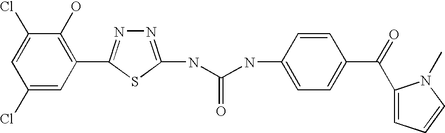 Intermediate 41-1

### (4-Amino-phenyl)-(1-methyl-1H-pyrrol-2-yl)-methanone

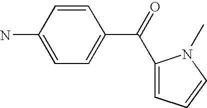

To 1 g of (4-nitro-phenyl)-(1-methyl-1H-pyrrol-2-yl)-methanone (prepared as in J. Med. Chem., 1997, vol. 40, no. 11, p. 1578-1584) dissolved in ethanol was added 4 g of tin chloride. The mixture was refluxed for 3 hours. The solvent was evaporated and water and concentrated NaOH solution were added. After extraction with dichloromethane and evaporation, 0.75 g of a white solid were obtained, which were used without further purification.

0.75 g of intermediate 41-1 and 500 mg of the product of Preparation 3 were dissolved in dioxane and the mixture was refluxed for 3 hours. The hot solution was filtered and the obtained precipitate was washed by acetone to give 350 mg of a product melting over 320° C.

Microanalysis: theory (%): C: 51.6; H: 3.10, N: 14.3; obtained (%): C: 51.4; H: 3.00, N: 14.2

### Example 42

### 4-(4-{3-[5-(3,5-Dichloro-2-hydroxy-phenyl)-[1,3,4]thiadiazol-2-yl]-ureido}-benzoyl)-benzoic Acid Ethyl Ester

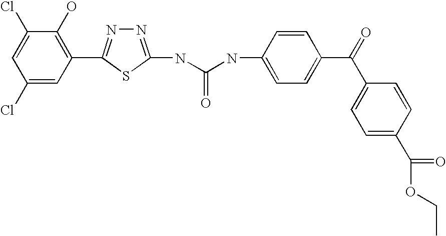 Intermediate 42-1

### 4-(4-Acetylamino-benzoyl)-benzoic Acid Methyl Ester

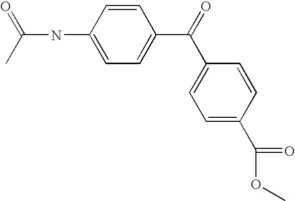 13.5 g of methyl 4-chlorocarbonylbenzoate were added to 9.2 g acetanilide dissolved in dichloroethane. 7 g of AlCl₃ were added portionwise and the mixture was refluxed for 3 hours. After return to RT, it was poured on a mixture of ice and water and the obtained precipitate was washed with acetone to give 5.2 g of a product melting at 212° C.
Intermediate 42-2

### 4-(4-Amino-benzoyl)-benzoic Acid

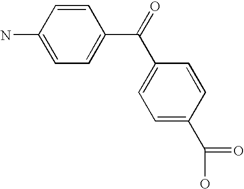 5.2 g of intermediate 42-1 were dissolved in ethanol, 10 ml of concentrated NaOH were added and the mixture was refluxed for 1 hour. HCl was added and the solvents were evaporated to give 3.2 g of a white powder used as such in the next step.
Intermediate 42-3

### 4-(4-Amino-benzoyl)-benzoic Acid Ethyl Ester

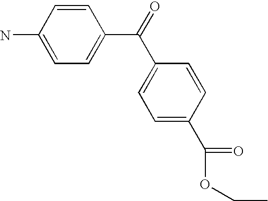 3.2 g of intermediate 42-2 were dissolved in ethanol, 5 ml of concentrated sulfuric acid were added and the mixture was refluxed for 6 hours. Ethanol was evaporated and a water solution of hydrogenocarbonate was added. The resulting mixture was extracted by dichloromethane, the dichloromethane was evaporated and the residue crystallized in isopropyl ether to give 1.8 g of a product melting at 125° C.

1.7 g of intermediate 42-3 and 2.1 g of the product of Preparation 3 were dissolved in dioxane and the mixture was refluxed for 6 hours. The hot solution was filtered to give a precipitate which was recrystallized in DMF and washed by acetone and methanol to give 470 mg of a product melting over 320° C.

LCMS: Rt: 4.5 mn; M/z: 557; Purity: 97%

### Example 43

### 3-{3-[5-(3,5-Dichloro-2-hydroxy-phenyl)-[1,3,4]thiadiazol-2-yl]-ureido}-N-methyl-N-phenyl-benzamide

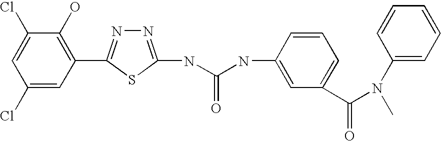 Intermediate 43-1

### 3-Amino-N-methyl-N-phenyl-benzamide

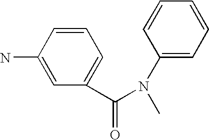 2 g of 4-nitrobenzoic acid-(N-methyl-anilide), prepared as in CA 1949, 4240, were dissolved in methanol and 5% palladium on charcoal wetted by methoxyethanol were added. This mixture was submitted to hydrogenation during 4 hours at 1 atmospher, the catalysts was filtered out and the solvent evaporated. The obtained residue was crystallized in pentane to give 600 mg of an off-white solid used as such in the next step.

430 mg of intermediate 43-1 and 520 mg of the product of Preparation 3 were dissolved in dioxane and the mixture was refluxed for 6 hours. The hot solution was filtered to give a precipitate which washed by dilute HCl, acetone and ethanol to give 85 mg of a product melting over 320° C.

Microanalysis: theory (%, 1H₂O): C: 51.9; H: 3.60, N: 13.1; obtained (%): C: 52.2, H: 3.40; N: 13.2

### Example 44

### N-Butyl-3-{3-[5-(3,5-dichloro-2-hydroxy-phenyl)-[1,3,4]thiadiazol-2-yl]-ureido}-N-phenyl-benzamide

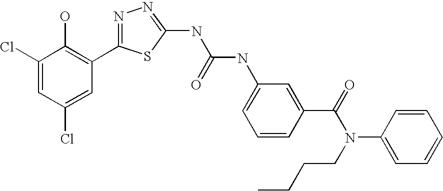 Intermediate 44-1

### N-Butyl-3-nitro-N-phenyl-benzamide

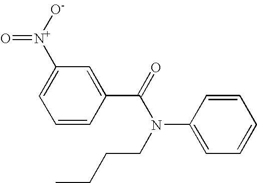 5 ml of N-butylaniline were dissolved in ethyl acetate and 4.6 ml of triethylamine were added. 5.6 g of 3-nitrobenzoyl chloride dissolved in ethyl acetate were added dropwise and the reaction mixture was stirred for one night at RT. It was then filtered and the filtrate was evaporated and purified by chromatography on a silica gel column using cyclohexane/AcOEt (85/15 as eluant to give 1 g of the expected product
Intermediate 44-2

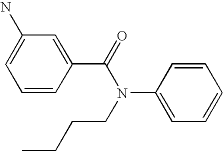

### 3-Amino-N-butyl-N-phenyl-benzamide

1 g of intermediate 44-1 were dissolved in ethyl acetate and 5% palladium on charcoal wetted by ethyl acetate were added. This mixture was submitted to hydrogenation during 4 hours at 1 atm., the catalysts was filtered out and the solvent evaporated. The obtained residue was crystallized in pentane to give 700 mg of an off-white solid used as such in the next step.

158 mg of intermediate 44-2 and 113.4 mg of the product of Preparation 3 were dissolved in dioxane and the mixture was refluxed for 6 hours. The hot solution was filtered to give a precipitate which washed by dilute HCl, acetone and ethanol to give 56 mg of a product melting over 320° C.

LCMS: Rt: 5.07 mn; M/z: 557; Purity: 96%

### Example 45

### 1-[5-(3,5-Dichloro-2-hydroxy-phenyl)-[1,3,4]thiadiazol-2-yl]-3-[4-(2-methyl-2H-tetrazol-5-yl)-phenyl]-urea

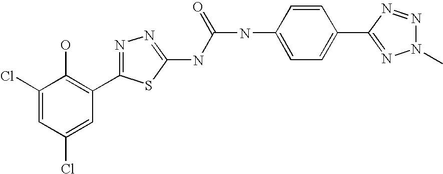 Intermediate 45-1

### 4-(2-Methyl-2H-tetrazol-5-yl)-phenylamine

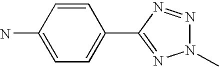 950 mg of 2-methyl-5-(4-nitrophenyl)-2H-tetrazole (prepared as described in Can. J. Chem., 1968 p. 2855) were dissolved in ethanol and 5 g of tin chloride were added. The mixture was refluxed for 4 hours and ethanol was evaporated. The residue was taken in dilute sodium hydroxide and extracted by dichloromethane to give 0.65 g of the expected product.

330 mg of intermediate 45-1 and 600 mg of the product of Preparation 3 were dissolved in dioxane and the mixture was refluxed for 7 hours. The hot solution was filtered to give a precipitate which washed by dilute HCl and methanol to give 88 mg of a product melting over 320° C.

Microanalysis: theory (%): C: 44.1; H: 2.62, N: 24.2; obtained (%): C: 44.1, H: 2.60; N: 24.3

### Example 46

### 1-[5-(3,5-Dichloro-2-hydroxy-phenyl)-[1,3,4]thiadiazol-2-yl]-3-[4-(4-dimethylamino-benzoyl)-phenyl]-urea

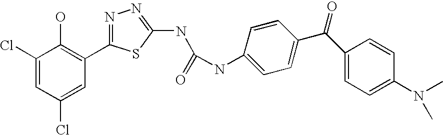 Intermediate 46-1

### (4-Amino-phenyl)-(4-dimethylamino-phenyl)-methanone

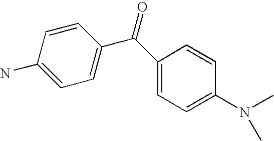 2 g of (4-dimethylamino-phenyl)-(4-nitro-phenyl)-methanone, prepared according to J. Chem. Soc., 1932 p. 642 were dissolved in ethanol and 8 g of tin chloride were added. The mixture was refluxed for 4 hours and ethanol was evaporated. The residue was taken in dilute sodium hydroxide and extracted by dichloromethane. The dichloromethane was evaporated and the residue dissolved in acetone. A solution of HCl in isopropyl ether was added and the precipitate was filtered and suspended in dilute NaOH to give 1 g of a product melting at 150° C.

500 mg of intermediate 46-1 and 500 mg of the product of Preparation 3 were dissolved in dioxane and the mixture was refluxed for 7 hours. The hot solution was filtered to give a precipitate which washed by dilute HCl, dilute ammonia and acetone to give 155 mg of a product melting over 320° C.

Microanalysis: theory (%, 1H₂O): C: 52.7; H: 3.87, N: 12.8; obtained (%): C: 52.7, H: 3.66; N: 12.8

### Example 47

### 1-[5-(3,5-Dichloro-2-hydroxy-phenyl)-[1,3,4]thiadiazol-2-yl]-3-[4-(4-[1,2,3]triazol-2-yl-benzoyl)-phenyl]-urea

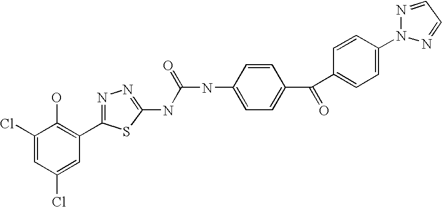 Intermediate 47-1

### (4-Nitro-phenyl)-(4-[1,2,3]triazol-2-yl-phenyl)-methanone

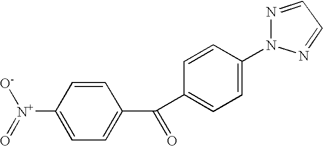 2.5 g of 1,2,3 triazole were dissolved in 50 ml of DMF and 1.4 g of NaH was added. The mixture was stirred at RT for 1 hour and 8.6 g of (4-Fluoro-phenyl)-(4-nitro-phenyl)-methanone dissolved in DMF were added. The reaction mixture was stirred at 80° C. for 4 hours. DMF was then evaporated and the residue was dissolved in dichloromethane and purify by chromatography on silica gel using dichloromethane/acetone 95/5 as eluant. A first spot was isolated, which after evaporation, gave 0.8 g of the expected product.

¹H NMR (DMSO-d₆): 8.47 (2H,d), 8.36 (4H,m), 8.05 (4H, m)

Intermediate 47-2

### (4-Amino-phenyl)-(4-[1,2,3]triazol-2-yl-phenyl)-methanone

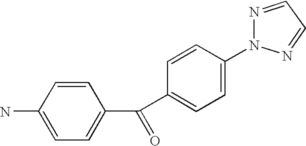 0.8 g of intermediate 47-1 were dissolved in ethanol and 3.2 g of tin chloride were added. The mixture was refluxed for 5 hours and ethanol was evaporated. The residue was taken in dilute sodium hydroxide and extracted by dichloromethane to give 0.3 g of the expected product used as such in the next step.

300 mg of intermediate 47-2 and 300 mg of the product of Preparation 3 were dissolved in dioxane and the mixture was refluxed for 7 hours. The hot solution was filtered to give a precipitate which washed by dilute HCl, dilute ammonia and acetone to give 180 mg of a product melting over 320° C.

Microanalysis: theory (%, 1H₂O): C: 50.5; H: 3.00, N: 17.2; obtained (%): C: 50.2, H: 2.80; N: 17.0

### Example 48

### 1-[5-(3.5-Dichloro-2-hydroxy-phenyl)-[1,3,4]thiadiazol-2-yl]-3-[4-(4-[1,2,3]triazol-1-yl-benzoyl)-phenyl]-urea

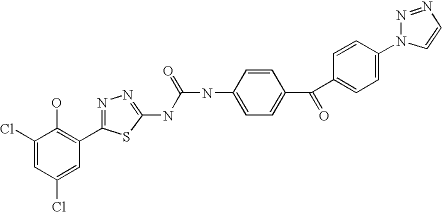 Intermediate 48-1

### (4-Nitro-phenyl)-(4-[1,2,3]triazol-1-yl-phenyl)-methanone

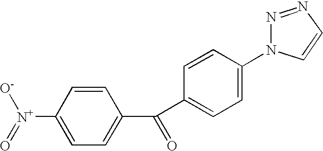

From the preparation of intermediate 47-1, during the chromatographic separation on silica gel using dichloromethane/acetone 95/5 as eluant, a second spot was isolated, which after evaporation, gave 3.3 g of the expected product.

¹H NMR (DMSO-d₆): 9.01(1H,d), 8.45 (2H,m), 8.18 (2H, d), 8.08 (1H,d), 8.04 (4H,m)

Intermediate 48-2

### (4-Amino-phenyl)-(4-[1,2,3]triazol-1-yl-phenyl)-methanone

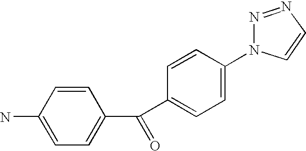

3.3 g of intermediate 48-1 were dissolved in ethanol and 13 g of tin chloride were added. The mixture was refluxed for 5 hours and ethanol was evaporated. The residue was taken in dilute sodium hydroxide and extracted by dichloromethane to give 0.6 g of the expected product used as such in the next step.

300 mg of intermediate 48-2 and 300 mg of the product of Preparation 3 were dissolved in dioxane and the mixture was refluxed for 7 hours. The hot solution was filtered to give a precipitate which washed by dilute HCl, dilute ammonia and acetone to give 180 mg of a product melting over 320° C.

Microanalysis: theory (%, 1H₂O): C: 50.5; H: 3.00, N: 17.2; obtained (%): C: 50.3, H: 2.90; N: 16.9

### Example 49

### 1-[5-(3,5-Dichloro-2-hydroxy-phenyl)-[1,3,4]thiadiazol-2-yl]-3-[4-(4-[1,2,4]triazol-1-yl-benzoyl)-phenyl]-urea

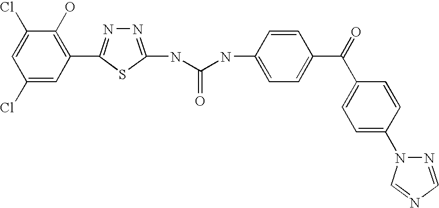 Intermediate 49-1

### (4-Nitro-phenyl)-(4-[1,2,4] triazol-1-yl-phenyl)-methanone

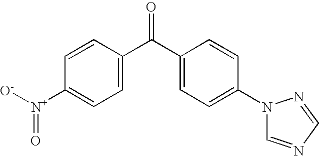 1.7 g of 1,2,4 triazole were dissolved in 50 ml of DMF and 1.0 g of NaH was added. The mixture was stirred at RT for 1 hour and 5 g of (4-Fluoro-phenyl)-(4-nitro-phenyl)-methanone dissolved in DMF were added. The reaction mixture was stirred at 80° C. for 4 hours. After return to RT, water was added and the obtained precipitate was dissolved in dichloromethane. A tar was eliminated and, after evaporation, the dichloromethane solution gave 3.2 g of an off-white product.

¹H NMR (DMSO-d6): 9.45 (1H,s), 8.40 (2H,d), 8.35 (1H, s), 8.12 (2H, d), 7.98 (4H,m)

Intermediate 49-2

### (4-Amino-phenyl)-(4-[1,2,4]triazol-1-yl-phenyl)-methanone

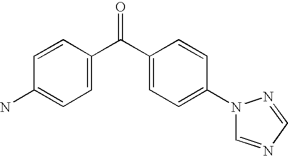 500 mg of intermediate 49-1 were dissolved in ethanol and 2 g of tin chloride were added. The mixture was refluxed for 5 hours and ethanol was evaporated. The residue was taken in dilute sodium hydroxide and extracted by dichloromethane to give 0.26 g of the expected product used as such in the next step.

52 mg of intermediate 49-2 and 55 mg of the product of Preparation 3 were dissolved in dioxane and the mixture was refluxed for 4 hours. The hot solution was filtered to give a precipitate which washed by dilute HCl, dilute ammonia and acetonitrile to give 41 mg of the expected product.

LCMS: Rt: 2.35 mn; M/z: 553; Purity: 86%

### Example 50

### 1-[5-(3,5-Dichloro-2-hydroxy-phenyl)-[1,3,4]thiadiazol-2-yl]-3-[4-(4-imidazol-1-yl-benzoyl)-phenyl]-urea

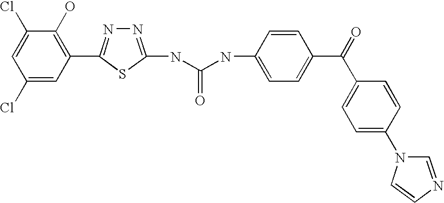 Intermediate 50-1

### (4-Imidazol-1-yl-phenyl)-(4-nitro-phenyl)-methanone

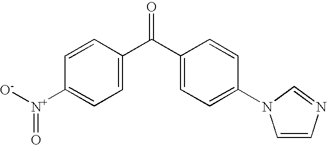

1.7 g of imidazole were dissolved in 50 ml of DMF and 1.0 g of NaH was added. The mixture was stirred at RT for 1 hour and 5 g of (4-Fluoro-phenyl)-(4-nitro-phenyl)-methanone dissolved in DMF were added. The reaction mixture was stirred at 80° C. for 4 hours. After return to RT, water was added and the obtained precipitate was washed by isopropyl ether to give 4.2 g of a white-off product.

¹H NMR (DMSO-d6): 8.39 (2H,d), 7.89 (2H,d), 7.87 (1H, s), 7.86 (2H, d), 7.55 (2H,d), 7.39 (1H,s)

Intermediate 50-2

### (4-Amino-phenyl)-(4-imidazol-1-yl-phenyl)-methanone

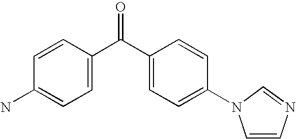

1 g of intermediate 50-1 were dissolved in methanol and 5% palladium on charcoal wetted by ethyl acetate were added. This mixture was submitted to hydrogenation during 3 hours at 1 atm., the catalysts was filtered out and the solvent evaporated. The obtained residue was purified by chromatography on a silica gel column using dichloromethane/methanol 90/10 as eluant to obtain 0.7 g of the expected product.

60 mg of intermediate 50-2 and 55 mg of the product of Preparation 3 were dissolved in dioxane and the mixture was refluxed for 4 hours. The hot solution was filtered to give a precipitate which washed by dilute HCl, dilute ammonia and methanol to give 44 mg of the expected product.

LCMS: Rt: 2.03 mn; M/z: 551; Purity: 87%

### Example 51

### 1-[5-(3,5-Dichloro-2-hydroxy-phenyl)-[1,3,4]thiadiazol-2-yl]-3-[4-(pyrrole-1-carbonyl)-phenyl]-urea

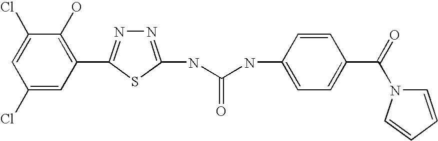 Intermediate 51-1

### (4-Amino-phenyl)-pyrrol-1-yl-methanone

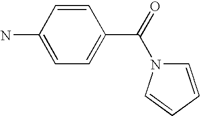 300 mg of 1-(4-nitro-benzoyl)-pyrrole, obtained according to J. Heterocycl. Chem., 2000, vol 37 no 1 p. 15-24 were dissolved in methanol, and 287 mg iron and 0.7 ml of concentrated HCl were added. The reaction mixture refluxed for 2 hours. After return to RT, the methanol was evaporated and water was added. After basification with sodium hydrogenocarbonate, a precipitate was obtained, which was dissolved in methanol. A tar was discarded and methanol evaporated to give 86 mg of expected product.

¹H NMR (DMSO-d₆): 7.56 (2H,d), 7.35 (2H,d), 6.68 (2H, d), 7.86(2H, d), 6.35 (2H,d)

40 mg of intermediate 51-1 were and 55 mg of the product of Preparation 3 were dissolved in dioxane and the mixture was refluxed for 4 hours. The hot solution was filtered to give a precipitate which was purified by preparative HPLC (Reverse phase HPLC, gradient from CH₃CN/H₂O/TFA: 5/95/0.05 to CH₃CN/H₂O/TFA: 80/20/0.05).

LCMS: Rt: 2.47 mn; M/z: 475; Purity: 100%

### Example 52

### 1-[4-(2-Acetyl-pyrrole-1-carbonyl)-phenyl]-3-[5-(3,5-dichloro-2-hydroxy-phenyl)-[1,3,4]thiadiazol-2-yl]-urea

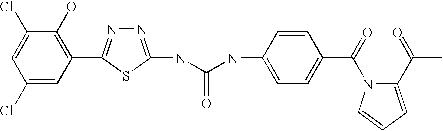 Intermediate 52-1

### 1-[1-(4-Nitro-benzoyl)-1H-pyrrol-2-yl]-ethanone

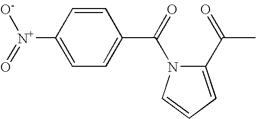 1.3 g of 4-nitrobenzoyl chloride was dissolved in dichloromethane and 1 ml TEA and 85.5 mg DMAP were added. 1.1 g of 2-acetyl-pyrrole was then added and the mixture was stirred at RT for 1 hour. Water was then added, the organic phase was separated and evaporated and the obtained residue was triturated in isopropyl ether to give 2 g of a brown solid.

¹H NMR (DMSO-d₆): 8.88 (2H,d), 7.82 (2H,d), 7.65 (1H, dd), 7.45 (1H, dd), 6.54 (1H, dd)

Intermediate 52-2

### 1-[1-(4-Amino-benzoyl)-1H-pyrrol-2-yl]-ethanone

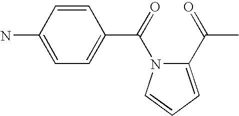 790 mg of intermediate 52-1 were dissolved in ethyl acetate and 5% palladium on charcoal wetted by ethyl acetate were added. This mixture was submitted to hydrogenation during 3 hours at 1 atm., the catalysts was filtered out and the solvent evaporated. The obtained residue was triturated in isopropyl ether to give 750 mg of a light yellow solid

¹H NMR (DMSO-d₆): 7.42 (1H,dd), 7.25 (2H,d), 7.21 (1H, dd), 6.52 (2H, d), 6.45 (1H, dd)

46 mg of intermediate 52-2 were dissolved in DCM and 23 mg triphosgene and 42 μl DIEA were added. The reaction mixture was stirred at RT for 30 nm, and 52 mg of the product of preparation 2 were added. The reaction mixture was stirred at RT for 3 hours, DCM was evaporated and the obtained precipitate was by dilute HCl and methanol to give 49 mg of a gray powder.

LCMS: Rt: 2.38 mn; M/z: 516; Purity: 100%

### Example 53

### 1-(4-{3-[5-(3,5-Dichloro-2-hydroxy-phenyl)-[1,3,4]thiadiazol-2-yl]-ureido}-benzoyl)-1H-pyrrole-2-carboxylic Acid Ethyl Ester

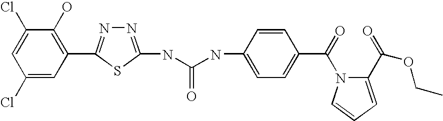 Intermediate 53-1

### 1-(4-Nitro-benzoyl)-1H-pyrrole-2-carboxylic Acid Ethyl Ester

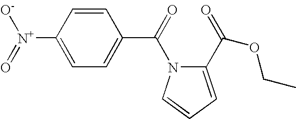 1.3 g of 4-nitrobenzoyl chloride was dissolved in dichloromethane and 1 ml TEA and 85.5 mg DMAP were added. 1.1 g of 2-ethoxycarbonyl-pyrrole was then added and the mixture was stirred at RT for 1 hour. Water was then added, the organic phase was separated and evaporated and the obtained residue was triturated in isopropyl ether to give 2 g of a brown solid.
Intermediate 53-2

### 1-(4-Amino-benzoyl)-1H-pyrrole-2-carboxylic Acid Ethyl Ester, Hydrochloride

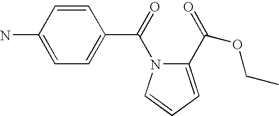 500 mg of intermediate 53-1 were dissolved in ethyl acetate and 5% palladium on charcoal wetted by ethyl acetate were added. This mixture was submitted to hydrogenation during 3 hours at 1 atm., the catalysts was filtered out and the solvent evaporated. The obtained residue was dissolved in isopropyl alcohol and hydrochloric acid in isopropyl alcohol was added to give 160 mg of a light yellow solid

¹H NMR (DMSO-d₆): 7.40 (2H,d), 7.05 (1H,m), 6.55 (2H, dd), 6.45 (1H, m), 6.30 (1H, m), 4.05 (2H, q), 1.15 (3H, t)

60 mg of intermediate 53-2 were dissolved in DCM and 23 mg triphosgene and 77 μl DIEA were added. The reaction mixture was stirred at RT for 30 nm, and 52 mg of the product of preparation 2 and 42 μl DIEA were added. The reaction mixture was stirred at RT for 3 hours, DCM was evaporated and the obtained precipitate was by dilute HCl and methanol to give 55 mg of a gray powder.

LCMS: Rt: 2.57 mn; M/z: 547; Purity: 100%

### Example 54

### 4-{3-[5-(2-Hydroxy-5-nitro-phenyl)-[1,3,4]thiadiazol-2-yl]-ureido}-benzoic Acid Ethyl Ester

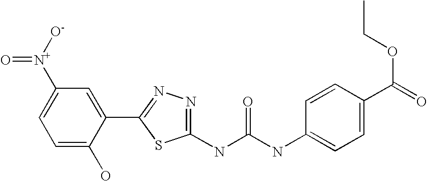 Intermediate 54-1

### 2-Hydroxy-5-nitro-benzoyl Chloride

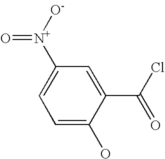 20 g of 5-nitro-salicylic acid were suspended in 120 ml dichloromethane and 35 ml of thionyl chloride and 0.5 ml of DMF were added. The mixture was refluxed until a clear solution was obtained and the solvent evaporated, the temperature being maintained below 40° C. The residue was triturated in ether to yield 22 g of a yellow solid used as such in the following step
Intermediate 54-2

### 2-Hydroxy-5-nitro-benzoyl Thiosemicarbazide

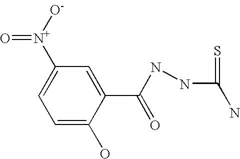 22 g of Intermediate 54-1 were dissolved in 50 ml THF and were added dropwise to 26.2 g of thiosemicarbazide suspended in 100 ml THF. The mixture was stirred at RT for 2 hours and the solvent was evaporated, the residue washed by 10% HCl water and acetone to give 22.6 g of a yellow solid used as such in the following step
Intermediate 54-3

### 2-(5-Amino-[1,3,4]thiadiazol-2-yl)-4-nitro-phenyl

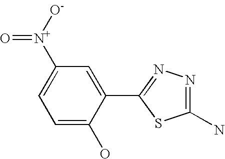

22.6 g of Intermediate 54-2 were suspended in 100 ml toluene and 8.6 ml of methane sulfonic acid were added. The mixture was refluxed for 5 hours. After return to RT, the obtained precipitate was filtered, washed with ammonia and acetone. The obtained residue was washed by hot methanol to give 8 g of an orange product.

¹H NMR (DMSO-d₆): 8.90 (1H, s), 8.26 (1H, d), 7.30 (2H,s), 7.15 (1H,d)

7.8 g of intermediate 54-3 and one equivalent of ethyl-4-isocyanatobenzoate were dissolved in THF and the solution was refluxed for 2 hours. The hot THF solution was filtered and the obtained precipitate was washed by hot methanol and acetone to give a yellow solid. 1 g of this solid was recrystallized in DMF and the obtained crystals were washed by acetone and methanol to give Example 54.

Microanalysis: theory (%): C: 50.3; H: 3.52, N: 16.3; obtained (%): C: 50.0, H: 3.45; N: 16.0

### Example 55

### 4-{3-[5-(2-Hydroxy-4-methoxy-phenyl)-[1,3,4]thiadiazol-2-yl]-ureido}-benzoic Acid Ethyl Ester

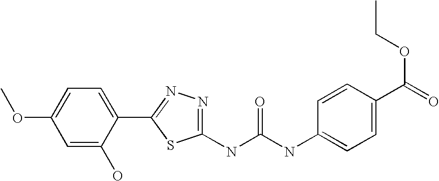 Intermediate 55-1

### 2-Hydroxy-4-methoxy-benzoyl Chloride

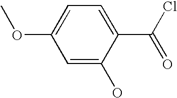 8 g of 4-methoxy-salicylic acid were suspended in 60 ml dichloromethane and 15 ml of thionyl chloride and 0.5 ml of DMF were added. The mixture was refluxed for 1 hour and the solvent evaporated, the temperature being maintained below 40° C. The residue was triturated in ether to yield 7.7 g of a white solid used as such in the following step.
Intermediate 55-2

### 2-Hydroxy-4-methoxy-benzoyl Thiosemicarbazide

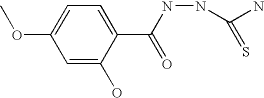

7.7 g of Intermediate 55-1 were dissolved in THF and were added dropwise to 9.4 g of thiosemicarbazide suspended in THF. After stirring 2 hours at RT, the solvent was evaporated, the residue washed by 10% HCl water and acetone to give 4.6 g of a white solid used as such in the following step.

Intermediate 55-3

### 2-(5-Amino-[1,3,4]thiadiazol-2-yl)-5-methoxy-phenyl

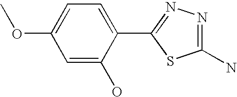

4.6 g of Intermediate 55-2 were suspended in 30 ml toluene and 1.8 ml of methane sulfonic acid were added. The mixture was refluxed for 5 hours. After return to RT, the obtained precipitate was filtered, washed with ammonia and acetone. The obtained residue was washed by hot methanol to give 1.2 g of a white product.

¹H NMR (DMSO-d₆): 7.65 (1H, d), 7.22(2H, s), 6.65 (1H,d), 6.60 (1H,s), 3.75 (3H,s)

1 g of Intermediate 55-3 and 0.86 g of ethyl-4-isocyanatobenzoate were dissolved in THF and the solution was refluxed for 2 hours. The hot THF solution was filtered and the obtained precipitate was washed by methanol and acetone to give 45 mg of a white solid.

Microanalysis: theory (%, 1.5H₂O): C: 51.7; H: 4.79, N: 12.7; obtained (%): C: 51.5, H: 4.60; N: 12.5

### Example 56

### 4-{3-[5-(2-Hydroxy-3-methoxy-phenyl)-[1,3,4]thiadiazol-2-yl]-ureido}-benzoic Acid Ethyl Ester

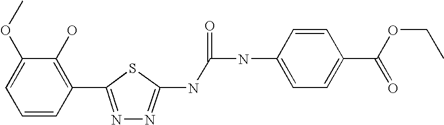 Intermediate 56-1

### 2-Hydroxy-3-methoxy-benzoyl Chloride

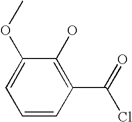

5 g of 3-methoxy-salicylic acid were suspended in 40 ml dichloromethane and 10 ml of thionyl chloride and 0.5 ml of DMF were added. The mixture was refluxed for 1 hour and the solvent evaporated, the temperature being maintained below 40° C., to give 5 g of a white solid which was used as such in the following step

Intermediate 56-2

### 2-Hydroxy-3-methoxy-benzoyl thiosemicarbazide

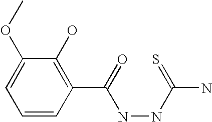

5 g of Intermediate 56-1 were dissolved in THF and were added dropwise to 6.8 g of thiosemicarbazide suspended in THF. After stirring 2 hours at RT, the solvent was evaporated, the residue washed by 10% HCl water and acetone to give 3.4 g of a white solid used as such in the following step

Intermediate 56-3

### 2-(5-Amino-[1,3,4]thiadiazol-2-yl)-6-methoxy-phenol

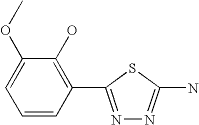 3.4 g of Intermediate 56-2 were suspended in 30 ml toluene and 1.8 ml of methane sulfonic acid were added. The mixture was refluxed for 5 hours. After return to RT, the obtained precipitate was filtered, washed with ammonia and acetone to give 2.5 g of a white product.

¹H NMR (DMSO-d₆): 7.47 (1H, d), 7.22(2H, s), 7.18 (1H,d), 6.80 (1H,t)

2.5 g of Intermediate 56-3 and 1.5 g of ethyl-4-isocyanatobenzoate were dissolved in THF and the solution was refluxed for 2 hours. The hot THF solution was filtered and the obtained precipitate was washed by acetone to give 2.4 g of a white solid.

Microanalysis: theory (%): C: 55.1; H: 4.38, N: 13.5; obtained (%): C: 54.9, H: 4.29; N: 13.5

### Example 57

### 4-{3-[5-(2-Hydroxy-6-methoxy-phenyl)-[1,3,4]thiadiazol-2-yl]-ureido}-benzoic Acid Ethyl Ester

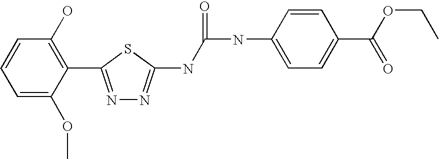 Intermediate 57-1

### 2-Hydroxy-6-methoxy-benzoyl Chloride

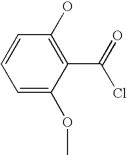 5 g of 6-methoxy-salicylic acid were suspended in 40 ml dichloromethane and 10 ml of thionyl chloride and 0.5 ml of DMF were added. The mixture was refluxed for 1 hour and the solvent evaporated, the temperature being maintained below 40° C., to give 5 g of a brown oil which was used as such in the following step.
Intermediate 57-2

### 2-Hydroxy-6-methoxy-benzoyl thiosemicarbazide

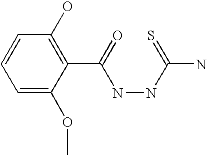 5 g of Intermediate 57-1 were dissolved in THF and were added dropwise to 6.8 g of thiosemicarbazide suspended in THF. After stirring 2 hours at RT, the solvent was evaporated, the residue washed by 10% HCl water and acetone to give 500 mg of a white solid used as such in the following step.
Intermediate 57-3

### 2-(5-Amino-[1,3,4]thiadiazol-2-yl)-3-methoxy-phenol

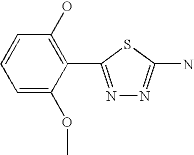 500 mg of Intermediate 57-2 were suspended in toluene and 0.2 ml of methane sulfonic acid were added. The mixture was refluxed for 6 hours. After return to RT, the obtained precipitate was filtered, washed with ammonia and acetone to give 0.19 g of a white product.

¹H NMR (DMSO-d₆): 7.27 (1H, t), 7.22(2H, s), 6.78 (1H,t), 6.76 (1H,t)

0.19 g of Intermediate 57-3 and 0.16 g of ethyl-4-isocyanatobenzoate were dissolved in THF and the solution was refluxed for 2 hours. The hot THF solution was filtered and the obtained precipitate was washed by acetone to give 160 mg of a white solid.

Microanalysis: theory (%, 1H₂O): C: 52.8; H: 4.66, N: 13.0; obtained (%): C: 53.0, H: 4.27; N: 13.2

### Example 58

### Ammonium, 2-{5-[3-(4-ethoxycarbonyl-phenyl)-ureido]-[1.3.41]thiadiazol-2-yl}-4.6-dinitro-phenolate

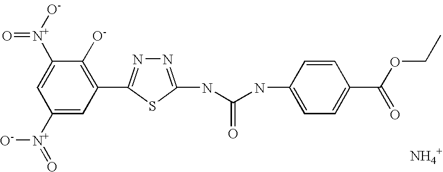 Intermediate 58-1

### 2-Hydroxy-3,5-dinitro-benzoyl Chloride

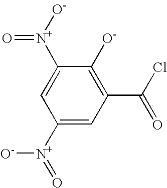 8 g of 3,5-dinitro-salicylic acid were suspended in 50 ml dichloromethane and 11 ml of thionyl chloride and 0.5 ml of DMF were added. The mixture was refluxed for 1 hour and the solvent evaporated, the temperature being maintained below 40° C., to give 8 g of a white powder which was used as such in the following step.
Intermediate 58-2

### 3,5-Dinitro-2-hydroxy-benzoyl thiosemicarbazide

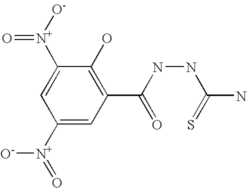 8 g of Intermediate 58-1 were dissolved in THF and were added dropwise to 9.6 g of thiosemicarbazide suspended in DMF. After stirring 2 hours at RT, water and dilute HCl were added and the mixture was extracted by ethyl acetate. The organic phase was then washed several times by dilute HCl, dried over magnesium sulfate and evaporated. The obtained residue was triturated in ethanol and washed with to give 2.3 g of an orange solid used as such in the following step.
Intermediate 58-3

### 2-(5-Amino-[1,3,4]thiadiazol-2-yl)-4,6-dinitro-phenyl

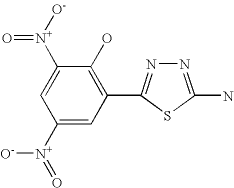 2.3 g of Intermediate 58-2 were suspended in toluene and 0.75 ml of methane sulfonic acid were added. The mixture was refluxed for 5 hours. After return to RT, the obtained precipitate was filtered, washed with ammonia and acetone to give 0.5 g of a gray product.

¹H NMR (DMSO-d₆): 8.78 (1H, s), 8.48 (1H, s), 7.44(2H, s)

0.5 g of Intermediate 57-3 and 0.34 g of ethyl-4-isocyanatobenzoate were dissolved in THF and the solution was refluxed for 2 hours. The hot THF solution was filtered and the precipitate washed by dilute ammonia, DMF and acetone to give 0.032 g of a red solid.

Microanalysis: theory (%): C: 43.9; H: 3.49, N: 19.9; obtained (%): C: 43.8, H: 3.34; N: 19.9

### Example 59

### 4-{3-[5-(5-Chloro-2-hydroxy-3-methoxy-phenyl)-[1,3,4]thiadiazol-2-yl]-ureido}-benzoic Acid Ethyl Ester

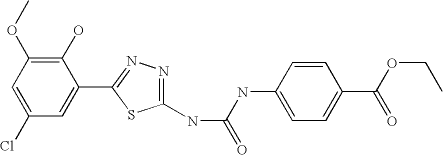 Intermediate 59-1

### 5-Chloro-2-hydroxy-3-methoxy-benzoic Acid Methyl Ester

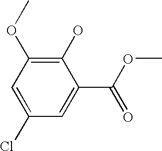 20 g of methyl-3-methoxy salicylate were dissolved in 200 ml toluene and 17 ml of sulfuryl chloride in toluene were added dropwise. The mixture was stirred at RT for 12 hours, then a dilute aqueous solution of sodium hydrogenocarbonate was slowly added and the organic phase was separated and evaporated. The obtained residue was crystallized in pentane to give 9.5 g of a white solid melting at 105° C.
Intermediate 59-2

### 5-Chloro-2-hydroxy-3-methoxy-benzoic Acid

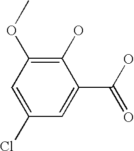 9.5 g of intermediate 59-1 were dissolved in 20 ml methanol and 3.51 g of sodium hydroxide and 50 ml water were added. The mixture was refluxed for 5 hours, methanol was then evaporated and the obtained solution was acidified by concentrated HCl. A precipitate was obtained which was washed by water and dichloromethane to give 7.9 g of a white solid used without further purification in the next step.
Intermediate 59-3

### 5-Chloro-2-hydroxy-3-methoxy-benzoyl chloride

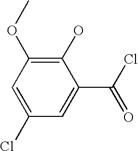 7.9 g of Intermediate 59-2 were suspended in 80 ml dichloromethane and 12.4 ml of thionyl chloride and 0.5 ml of DMF were added. The mixture was refluxed for 1 hour and the solvent evaporated, the temperature being maintained below 40° C., to give 8 g of a yellow powder which was used as such in the following step.
Intermediate 59-4

### 5-Chloro-2-hydroxy-3-methoxy-benzoyl thiosemicarbazide

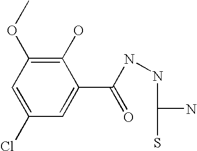 8 g of Intermediate 59-4 were dissolved in THF and were added dropwise to 10.6 g of thiosemicarbazide suspended in DMF. After stirring 2 hours at RT, the solvent was evaporated and the residue triturated in dilute HCl. The obtained precipitate was washed by ethanol and acetone to give 6.2 g of a yellow solid used as such in the next step.
Intermediate 59-5

### 2-(5-Amino-[1,3,4]thiadiazol-2-yl)-4-chloro-6-methoxy-phenol

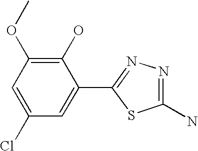 6.2 g of Intermediate 59-4 were suspended in toluene and 2.2 ml of methane sulfonic acid were added. The mixture was refluxed for 5 hours. After return to RT, the obtained precipitate was filtered, washed with ammonia and acetone to give 3.7 g of a yellow product

¹H NMR (DMSO-d₆): 10.5 (1H,s), 7.45 (1H, d), 7.22 (2H, s), 7.02 (1H, d)

0.5 g of Intermediate 59-5 and 0.37 g of ethyl-4-isocyanatobenzoate were dissolved in THF and the solution was refluxed for 2 hours. The hot THF solution was filtered and the precipitate washed by dilute ammonia and acetone to give 0.250 g of a white solid.

Microanalysis: theory (%): C: 50.8; H: 3.82, N: 12.5; obtained (%): C: 50.7, H: 3.86; N: 12.3

### Example 60

### 1-(4-Benzoyl-phenyl)-3-[5-(3,5-difluoro-2-hydroxy-phenyl)-[1,3,4]thiadiazol-2-yl]-urea

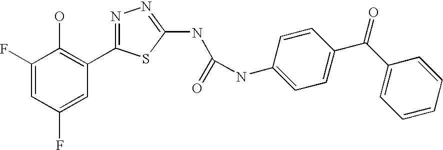 Intermediate 60-1

### 3,5-Difluoro-2-hydroxy-benzoyl Chloride

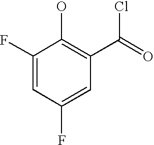 4 g of 3,5-difluoro-salicylic acid (prepared as described in Synth. Comm., 1996, 26, 14, p. 2775-2782) were suspended in 40 ml dichloromethane and 6.4 ml of thionyl chloride and 0.5 ml of DMF were added. The mixture was refluxed for 1 hour and the solvent evaporated, the temperature being maintained below 40° C., to give 4 g of an orange oil which was used as such in the following step.
Intermediate 60-2

### 3,5-Difluoro-2-hydroxybenzoyl thiosemicarbazide

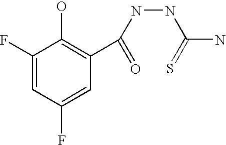 4 g of Intermediate 60-1 were dissolved in THF and were added dropwise to 6.4 g of thiosemicarbazide suspended in DMF. After stirring 4 hours at RT, the solvent was evaporated and the residue triturated in dilute HCl. The obtained precipitate was washed by dichloromethane and ethyl ether to give 2 g of an off-white solid used as such in the next step.
Intermediate 60-3

### 2-(5-Amino-[1,3,4]thiadiazol-2-yl)-4,6-difluoro-phenol

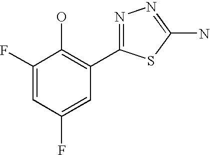 2 g of Intermediate 60-4 were suspended in toluene and 0.8 ml of methane sulfonic acid were added. The mixture was refluxed for 5 hours. Toluene was evaporated and dilute ammonia was added to the residue. Extraction by ethyl acetate gave 600 mg of a pale pink product.

¹H NMR (DMSO-d₆): 7.55(1H, m), 7.42(1H, m), 7.22(2H, s)

200 mg of amino-4-benzophenone were dissolved in DCM and 110 mg triphosgene and 0.23 ml DIEA were added. The reaction mixture was stirred at RT for 30 nm, and 250 mg of Intermediate 60-3 and 0.23 ml DIEA were added. The reaction mixture was stirred at RT for 12 hours, DCM was evaporated and the obtained precipitate was by dilute HCl and methanol to give 30 mg of a yellow solid.

Microanalysis: theory (%): C: 58.4; H: 3.12, N: 12.4; obtained (%): C: 58.1, H: 3.05; N: 12.3

### Example 61

### 4-{3-[5-(3,4,5-Trifluoro-2-hydroxy-phenyl)-[1,3,4]thiadiazol-2-yl]-ureido}-benzoic Acid Ethyl Ester

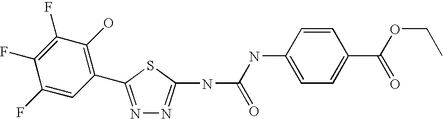 Intermediate 61-1

### 3,4,5-Trifluoro-2-hydroxy-benzaldehyde

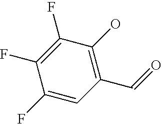 20 g of 2,3,4-Trifluorophenyl were dissolved in 120 ml of trifluoroacetic acid and 37.9 g of hexamethylenetetramine were added portionwise. The mixture was then heated at 100° C. for 3 hours. After return to RT, 180 ml of water and 45 ml of concentrated sulfuric acid were added. The mixture was stirred at RT for 2 hours and extracted with ethyl acetate, which, after evaporation gave 9 g of an orange oil used without further purification in the next step.
Intermediate 61-2

### 3,4,5-Trifluoro-2-hydroxy-benzoic Acid

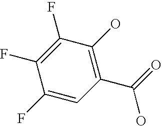 9 g of Intermediate 61-1 were dissolved in 150 ml dioxane and 27.8 g of sodium monophosphate and 7.3 g of sulfamic acid were added and the temperature was lowered to 10° C. 7.7 g of sodium sulfite dissolved in 7 ml water were slowly added and the mixture was stirred at 10° C. for 30 nm. After return to RT, the solution pH was adjusted to 2 by concentrated HCl and dioxane was evaporated. The residue was triturated in ethyl acetate. Evaporation of ethyl acetate gave 4 g of a brown solid used as such in the next step.
Intermediate 61-3

### 3,4,5-Trifluoro-2-hydroxy-benzoyl Chloride

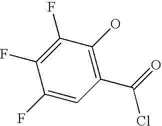 4 g of Intermediate 61-2 were suspended in 40 ml dichloromethane and 6.6 ml of thionyl chloride and 0.5 ml of DMF were added. The mixture was refluxed for 1 hour and the solvent evaporated, the temperature being maintained below 40° C., to give 4 g of an brown oil which was used as such in the following step.
Intermediate 61-4

### 3,4,5-Trifluoro-2-hydroxybenzoyl Thiosemicarbazide

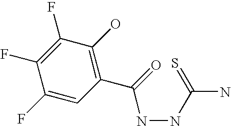 4 g of Intermediate 61-4 were dissolved in THF and were added dropwise to 5.7 g of thiosemicarbazide suspended in DMF. After stirring 4 hours at RT, the solvent was evaporated and the residue triturated in dilute HCl. The obtained precipitate was dissolved in ethyl acetate and dried over sodium sulfate. Ethyl acetate was evaporated in the residue crystallized in ethyl ether to give 1.7 g of a light brown solid used as such in the next step.
Intermediate 61-5

### 6-(5-Amino-[1,3,4]thiadiazol-2-yl)-2,3,4-trifluoro-phenol

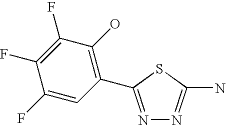 1.7 g of Intermediate 61-4 were suspended in toluene and 0.6 ml of methane sulfonic acid were added. The mixture was refluxed for 4 hours. Toluene was evaporated and dilute ammonia was added to the residue. The ammonia solution was acidified by bubbling sulfur dioxide to give a solid which was filtered and dissolved in ethyl acetate and dried over magnesium sulfate. Evaporation of ethyl acetate and trituration in isopropyl ether gave 0.58 g of a light brown solid.

¹H NMR (DMSO-d₆): 7.65(1H, t), 7.42(2H, s)

0.25 g of Intermediate 61-5 and 0.20 g of ethyl-4-isocyanatobenzoate were dissolved in THF and the solution was refluxed for 2 hours. The hot THF solution was filtered and the precipitate washed by acetone to give 0.170 g of a white solid melting at 309° C.

Microanalysis: theory (%): C: 49.3; H: 2.99, N: 12.8; obtained (%): C: 49.3, H: 2.99; N: 12.8

### Example 62

### 1-(4-Benzoyl-phenyl)-3-[5-(2,3,5-trichloro-6-hydroxy-phenyl)-[1,3,4]thiadiazol-2-yl]-urea

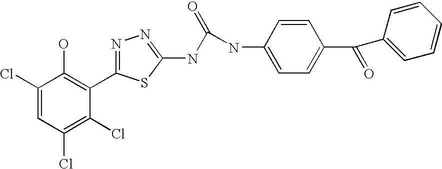 Intermediate 62-1

### 2,3,5-Trichloro-6-hydroxy-benzoyl Chloride

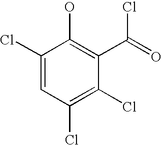 10 g of 3,5,6-trichloro-salicylic acid were suspended in 100 ml dichloromethane and 13 ml of thionyl chloride and 0.5 ml of DMF were added. The mixture was refluxed for 1 hour and the solvent evaporated, the temperature being maintained below 40° C., to give 10 g of a white solid which was used as such in the following step.
Intermediate 62-2

### 3,5,6-Trifluoro-2-hydroxybenzoyl Thiosemicarbazide

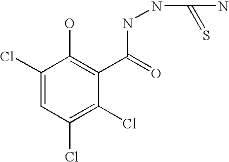 10 g of Intermediate 62-2 were dissolved in THF and were added dropwise to 11.3 g of thiosemicarbazide suspended in DMF. After stirring 4 hours at RT, the solvent was evaporated and the residue triturated in dilute HCl. The obtained precipitate was dissolved in 150 ml of hot methanol, a precipitate was discarded and the methanol was concentrated to half its initial volume. 2.5 g of a precipitate was then filtered out and used as such in the next step.
Intermediate 62-3

### 2-(5-Amino-[1,3,4]thiadiazol-2-yl)-3,4,6-trichloro-phenol

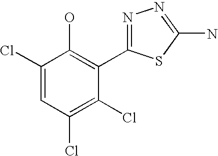 1 g of Intermediate 62-2 were dissolved in 5 g of polyphosphoric acid and the mixture was refluxed for 3 hours at 120° C. After return to RT, water was added and the mixture was adjusted to pH 7 with ammonia. The mixture was then extracted with ethyl acetate. After drying on magnesium sulfate, the ethyl acetate was evaporated and the residue triturated in isopropyl ether to give 200 mg of an off-white product.

¹H NMR (DMSO-d₆): 7.85 (1H, s), 7.65 (2H, s)

80 mg of amino-4-benzophenone were dissolved in DCM and 33 mg triphosgene and 0.07 ml DIEA were added. The reaction mixture was stirred at RT for 30 mn, and 100 mg of Intermediate 62-3 and 0.07 ml DIEA were added. The reaction mixture was stirred at RT for 12 hours, DCM was evaporated and the obtained precipitate was by dilute HCl and methanol. The obtained solid was then recrystallized in acetone to give 0.063 g of a yellow solid.

Microanalysis: theory (%): C: 50.8; H: 2.52, N: 10.8; obtained (%): C: 50.6, H: 2.32; N: 10.7

1. A method of treating a disorder associated with high levels of PAI-1 comprising administering to a patient in need thereof a therapeutically effective amount of at least one compound of formula (I), 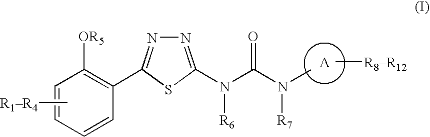
or a pharmaceutically-acceptable salt, prodrug, stereoisomer or solvate thereof, wherein:
A is aryl or heteroaryl;
R₁-R₄ and R₈-R₁₂ are independently selected from hydrogen, halogen, nitro, cyano, alkyl, substituted alkyl, aryl, heteroaryl, cycloalkyl, heterocyclo, —OR₁₃, —SR₁₄, —OC(═O)R₁₃, —C(═O)R₁₃, —CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅;
or any two of R₁-R₄ and R₈-R₁₂ located on neighboring atoms of the ring to which they are attached may be taken together to form a fused ring system in combination with the ring, wherein the fused ring system may be optionally further substituted;
R₅ is hydrogen, alkyl or substituted alkyl;
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and/or R₁₆ is selected independently.

2. The method according to claim 1, comprising administering to a patient in need thereof a therapeutically effective amount of at least one compound having the formula (Ia), 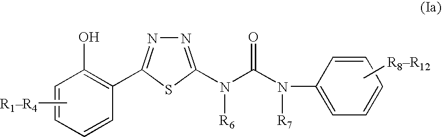
or a pharmaceutically-acceptable salt, prodrug, stereoisomer or solvate thereof, wherein:
R₁-R₄ and R₈—R₁₂ are independently selected from hydrogen, halogen, nitro, cyano, alkyl, substituted alkyl, cycloalkyl, aryl, heteroaryl, heterocyclo, —OR₁₃, —SR₁₄, —OC(═O)R₁₃, —CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃ and —NR₁₆C(═O)NR₁₄R₁₅;
or any two of R₁-R₅ and R₈-R₁₂ located on neighboring atoms of the ring to which they are attached may be taken together to form a fused ring system in combination with the ring, wherein the fused ring system may be optionally further substituted;

3. A method according to claim 2, or a pharmaceutically acceptable salt, prodrug or solvate thereof, in which:
R₁-R₄ are independently selected from hydrogen, halogen, nitro, cyano, alkyl, substituted alkyl, —OR₁₃ and —SR₁₃; and
R₈-R₁₂ are independently selected from hydrogen, halogen, nitro, cyano, alkyl, substituted alkyl, aryl, heteroaryl, cycloalkyl, heterocyclo, —OR₁₃, —SR₁₃, —OC(═O)R₁₃, —C(═O)R₁₃, —CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅, provided that when any one of R₈-R₁₂ is —NR₁₆C(═O)NR₁₄R₁₅, neither R₁₄ or R₁₅ are thiadiazole;
or any two of R₈-R₁₂ located on neighboring atoms of the ring may be taken together to form a fused ring system in combination with the ring, said fused ring system being optionally substituted.

4. A method according to claim 3, comprising administering to a patient in need thereof a therapeutically effective amount of at least one compound, or a pharmaceutically-acceptable salt, prodrug, stereoisomer or solvate thereof, wherein:
R₈-R₁₂ are selected from
(a) hydrogen, nitro, cyano, halogen, —OR₁₃, —C(═O)R₁₃, —CO₂R₁₃, —C(═O)NR₁₄R₁₅, —SR₁₃, —S(═O)R₁₃, —SO₂R₁₃, and —SO₂NR₁₄R₁₅; or
(b) optionally substituted heterocyclo and heteroaryl; or
(c) any two of R₈-R₁₂ located on neighboring atoms of the ring may be taken together to form a fused ring system in combination with the ring, said fused ring system being optionally substituted.

5. A method according to claim 3, comprising administering to a patient in need thereof a therapeutically effective amount of at least one compound having formula (Ib) 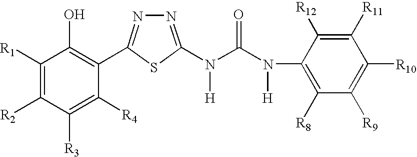
or a pharmaceutically-acceptable salt, prodrug, stereoisomer or solvate thereof, wherein:
R₁-R₄ are selected from hydrogen, halogen, nitro and C₁₋₆alkoxy.

6. A method according to claim 5, comprising administering to a patient in need thereof a therapeutically effective amount of at least one compound of formula (Ib), or a pharmaceutically-acceptable salt, prodrug, stereoisomer or solvate thereof, wherein:
R₁ and R₃ are halogen; and
R₂ and R₄ are hydrogen.

7. A compound having the formula (Ia) 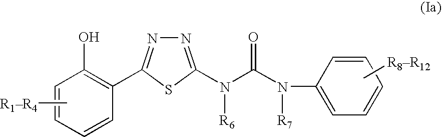
or a pharmaceutically-acceptable salt, prodrug, stereoisomer or solvate thereof, wherein:
R₁-R₄ and R₈-R₁₂ are independently selected from hydrogen, halogen, nitro and cyano, alkyl, substituted alkyl, aryl, heteroaryl, cycloalkyl, heterocyclo, —OR₁₃, —SR₁₃, —OC(═O)R₁₃, —C(═O)R₁₃—CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅; provided that when any one of R₈-R₁₂ is —NR₁₆C(═O)NR₁₄R₁₅, neither R₁₄ or R₁₅ are thiadiazole;
or any two of R₁-R₄ and R₈-R₁₂ located on neighboring atoms of the ring to which they are attached may be taken together to form a fused ring system in combination with the ring, wherein the fused ring system may be optionally further substituted;
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and R₁₆ is selected independently.

8. A compound according to claim 7, or a pharmaceutically-acceptable salt, prodrug, stereoisomer or solvate thereof, wherein:
R₁-R₄ are independently selected from hydrogen, halogen, nitro, cyano, alkyl, substituted alkyl, —OR₁₃ and —SR₁₃; and
R₈-R₁₂ are independently selected from hydrogen, halogen, nitro, cyano, alkyl, substituted alkyl, aryl, heteroaryl, cycloalkyl, heterocyclo, —OR₁₃, —SR₁₃, —OC(═O)R₁₃, —C(═O)R₁₃—CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅, provided that when any one of R₈-R₁₂ is —NR₁₆C(═O)NR₁₄R₁₅, neither R₁₄ or R₁₅ are thiadiazole;
or any two of R₈-R₁₂ located on neighboring atoms of the ring may be taken together to form a fused ring system in combination with the ring, said fused ring system being optionally substituted.

9. A compound according to claim 8, or a pharmaceutically-acceptable salt, prodrug, stereoisomer or solvate thereof, wherein:
R₈-R₁₂ are selected from
(a) hydrogen, nitro, cyano, halogen, —OR₁₃, —C(═O)R₁₃, —CO₂R₁₃, —C(═O)NR₁₄R₁₅, —SR₁₃, —S(═O)R₁₃, —SO₂R₁₃ and —SO₂NR₁₄R₁₅; or
(b) C₁₋₆alkyl, C₁₋₆alkyl, substituted heterocyclo and heteroaryl; or
(c) any two of R₈-R₁₂ located on neighboring atoms of the ring may be taken together to form a fused ring system in combination with the ring, said fused ring system being optionally substituted;
R₁₃ is hydrogen, alkyl, substituted alkyl, cycloalkyl, aryl, heteroaryl or heterocyclo; and
R₁₄, and R₁₅ are independently selected from hydrogen, alkyl, substituted alkyl, aryl, heteroaryl, cycloalkyl and heterocyclo.

10. A compound of claim 8 having the formula (Ib), 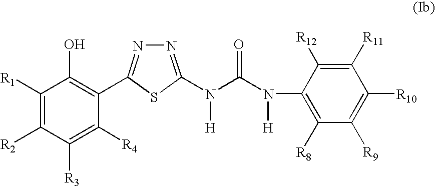
or a pharmaceutically-acceptable salt, prodrug, stereoisomer or solvate thereof.

11. A compound of claim 10, or a pharmaceutically-acceptable salt, prodrug, stereoisomer or solvate thereof, wherein
R₉ and R₁₀ are taken together to form a fused ring system in combination with the phenyl ring, said fused ring system substituted where valence allows with a group selected from hydrogen, oxo, nitro, cyano, halogen, C₁₋₆alkyl, substituted C₁₋₆alkyl, C₁₋₆alkyloxy and C₁₋₆haloalkyloxy.

12. A compound of claim 10, or a pharmaceutically-acceptable salt, prodrug, stereoisomer or solvate thereof, wherein:
R₁₀ is —C(═O)R₁₃; and
R₁₃ is aryl or heteroaryl substituted with a group selected from hydrogen, amino, nitro, cyano, halogen, aryl, heteroaryl, C₁₋₆alkyl, substituted C₁₋₆alkyl, C₁₋₆alkyloxy and C₁₋₆haloalkyloxy, C(═O)R₁₇, CO₂R₁₇, and —OR₁₇; and
R₁₇ and R₁₈ are independently hydrogen, C₁₋₆alkyl or phenyl.

13. A compound of claim 10, or a pharmaceutically-acceptable salt, prodrug, stereoisomer or solvate thereof, wherein:
R₁₁, is —C(═O)NR₁₄R₁₅;
R₁₄ is hydrogen or C₁₋₆alkyl;
R₁₅ is aryl or heteroaryl substituted with a group selected from hydrogen, amino, nitro, cyano, halogen, aryl, heteroaryl, C₁₋₆alkyl, substituted C₁₋₆alkyl, C₁₋₆alkyloxy and C₁₋₆haloalkyloxy, C(═O)R₁₇, CO₂R₁₇, and —OR₁₇; and
R₁₇ and R₁₈ are independently hydrogen or C₁₋₆alkyl.

14. A compound of claim 10, or a pharmaceutically-acceptable salt, prodrug, stereoisomer or solvate thereof, wherein
R₁-R₄ are selected from hydrogen, halogen, nitro and C₁₋₆alkoxy.

15. A compound of claim 14, or a pharmaceutically-acceptable salt, prodrug, stereoisomer or solvate thereof, wherein
R₁ and R₃ are halogen; and
R₂ and R₄ are hydrogen.

16. A compound selected from
(i) 1-[5-(3,5-Dichloro-2-hydroxy-phenyl)-[1,3,4]thiadiazol-2-yl]-3-(4-nitro-phenyl)-urea;
4-{3-[5-(3,5-Dichloro-2-hydroxy-phenyl)-[1,3,4]thiadiazol-2-yl]-ureido}-benzoic acid ethyl ester;
3-{3-[5-(3,5-Dichloro-2-hydroxy-phenyl)-[1,3,4]thiadiazol-2-yl]-ureido}-benzoic acid methyl ester;
4-{3-[5-(3,5-Dichloro-2-hydroxy-phenyl)-[1,3,4]thiadiazol-2-yl]-ureido}-benzoic acid methyl ester;
3-{3-[5-(3,5-Dichloro-2-hydroxy-phenyl)-[1,3,4]thiadiazol-2-yl]-ureido}-benzoic acid;
3-{3-[5-(3,5-Dichloro-2-hydroxy-phenyl)-[1,3,4]thiadiazol-2-yl]-ureido}-N-phenyl-benzamide;
3-{3-[5-(3,5-Dichloro-2-hydroxy-phenyl)-[1,3,4]thiadiazol-2-yl]-ureido}-N-(4-dimethylamino-phenyl)-benzamide;
3-{3-[5-(3,5-Dichloro-2-hydroxy-phenyl)-[1,3,4]thiadiazol-2-yl]-ureido}-N-(3-hydroxymethyl-phenyl)-benzamide;
3-{3-[5-(3,5-Dichloro-2-hydroxy-phenyl)-[1,3,4]thiadiazol-2-yl]-ureido}-N-(2-methoxy-phenyl)-benzamide;
3-{3-[5-(3,5-Dichloro-2-hydroxy-phenyl)-[1,3,4]thiadiazol-2-yl]-ureido}-N-(4-methoxy-phenyl)-benzamide;
3-{3-[5-(3,5-Dichloro-2-hydroxy-phenyl)-[1,3,4]thiadiazol-2-yl]-ureido}-N-m-tolyl-benzamide;
3-{3-[5-(3,5-Dichloro-2-hydroxy-phenyl)-[1,3,4]thiadiazol-2-yl]-ureido}-N-(4-pentyloxy-phenyl)-benzamide;
3-{3-[5-(3,5-Dichloro-2-hydroxy-phenyl)-[1,3,4]thiadiazol-2-yl]-ureido}-N-(1H-indol-5-yl)-benzamide;
3-{3-[5-(3,5-Dichloro-2-hydroxy-phenyl)-[1,3,4]thiadiazol-2-yl]-ureido}-N-(4-phenoxy-phenyl)-benzamide;
1-[5-(3,5-Dichloro-2-hydroxy-phenyl)-[1,3,4]thiadiazol-2-yl]-3-(3-oxo-1,3-dihydro-isobenzofuran-5-yl)-urea;
1-(2-Benzoyl-4-nitro-phenyl)-3-[5-(3,5-dichloro-2-hydroxy-phenyl)-[1,3,4]thiadiazol-2-yl]-urea;
1-[5-(3,5-Dichloro-2-hydroxy-phenyl)-[1,3,4]thiadiazol-2-yl]-3-(4-hydroxy-phenyl)-urea;
1-(4-Benzotriazol-2-yl-phenyl)-3-[5-(3,5-dichloro-2-hydroxy-phenyl)-[1,3,4]thiadiazol-2-yl]-urea;
1-[4-(4-Bromo-1-methyl-1H-pyrazol-3-yl)-phenyl]-3-[5-(3,5-dichloro-2-hydroxy-phenyl)-[1,3,4]thiadiazol-2-yl]-urea;
1-[5-(3,5-Dichloro-2-hydroxy-phenyl)-[1,3,4]thiadiazol-2-yl]-3-(4-imidazol-1-yl-phenyl)-urea;
5-(4-{3-[5-(3,5-Dichloro-2-hydroxy-phenyl)-[1,3,4]thiadiazol-2-yl]-ureido}-phenyl)-4-methyl-furan-3-carboxylic acid ethyl ester;
1-[5-(3,5-Dichloro-2-hydroxy-phenyl)-[1,3,4]thiadiazol-2-yl]-3-(4-[1,2,4]triazol-1-yl-phenyl)-urea;
1-[5-(3,5-Dichloro-2-hydroxy-phenyl)-[1,3,4]thiadiazol-2-yl]-3-(9-oxo-9H-fluoren-3-yl)-urea;
1-[5-(3,5-Dichloro-2-hydroxy-phenyl)-[1,3,4]thiadiazol-2-yl]-3-[4-(4-methoxy-benzoyl)-phenyl]-urea;
1-[5-(3,5-Dichloro-2-hydroxy-phenyl)-[1,3,4]thiadiazol-2-yl]-3-(1-oxo-1,3-dihydro-isobenzofuran-5-yl)-urea;
1-[5-(3,5-Dichloro-2-hydroxy-phenyl)-[1,3,4]thiadiazol-2-yl]-3-(2-methoxy-5-nitro-phenyl)-urea;
1-[5-(3,5-Dichloro-2-hydroxy-phenyl)-[1,3,4]thiadiazol-2-yl]-3-(2-fluoro-5-nitro-phenyl)-urea;
4-{3-[5-(3,5-Dichloro-2-hydroxy-phenyl)-[1,3,4]thiadiazol-2-yl]-ureido}-3-methyl-benzoic acid ethyl ester;
1-(4-Benzoyl-phenyl)-3-[5-(3,5-dichloro-2-hydroxy-phenyl)-[1,3,4]thiadiazol-2-yl]-urea;
1-[5-(3,5-Dichloro-2-hydroxy-phenyl)-[1,3,4]thiadiazol-2-yl]-3-[4-(pyridine-4-carbonyl)-phenyl]-urea;
Hydrochloride of 1-[5-(3,5-Dichloro-2-hydroxy-phenyl)-[1,3,4]thiadiazol-2-yl]-3-[4-(pyridine-3-carbonyl)-phenyl]-urea;
1-[5-(3,5-Dichloro-2-hydroxy-phenyl)-[1,3,4]thiadiazol-2-yl]-3-[4-(4-methyl-benzoyl)-phenyl]-urea;
1-[5-(3,5-Dichloro-2-hydroxy-phenyl)-[1,3,4]thiadiazol-2-yl]-3-[4-(4-fluoro-benzoyl)-phenyl]-urea;
1-[5-(3,5-Dichloro-2-hydroxy-phenyl)-[1,3,4]thiadiazol-2-yl]-3-[4-(2-fluoro-benzoyl)-phenyl]-urea;
1-[5-(3,5-Dichloro-2-hydroxy-phenyl)-[1,3,4]thiadiazol-2-yl]-3-(9-oxo-9,10-dihydro-acridin-3-yl)-urea;
1-[5-(3,5-Dichloro-2-hydroxy-phenyl)-[1,3,4]thiadiazol-2-yl]-3-(10-methyl-9-oxo-9,10-dihydro-acridin-3-yl)-urea;
1-[5-(3,5-Dichloro-2-hydroxy-phenyl)-[1,3,4]thiadiazol-2-yl]-3-(5-oxo-5, 10-dihydro-benzo[b][1,8]naphthyridin-8-yl)-urea;
1-[5-(3,5-Dichloro-2-hydroxy-phenyl)-[1,3,4]thiadiazol-2-yl]-3-[4-(thiophene-2-carbonyl)-phenyl]-urea;
(3-{3-[5-(3,5-Dichloro-2-hydroxy-phenyl)-[1,3,4]thiadiazol-2-yl]-ureido}-9-oxo-9H-acridin-10-yl)-acetic acid ethyl ester;
1-[5-(3,5-Dichloro-2-hydroxy-phenyl)-[1,3,4]thiadiazol-2-yl]-3-[4-(1H-pyrrole-2-carbonyl)-phenyl]-urea;
1-[5-(3,5-Dichloro-2-hydroxy-phenyl)-[1,3,4]thiadiazol-2-yl]-3-[4-(1-methyl-1H-pyrrole-2-carbonyl)-phenyl]-urea;
4-(4-{3-[5-(3,5-Dichloro-2-hydroxy-phenyl)-[1,3,4]thiadiazol-2-yl]-ureido}-benzoyl)-benzoic acid ethyl ester;
3-{3-[5-(3,5-Dichloro-2-hydroxy-phenyl)-[1,3,4]thiadiazol-2-yl]-ureido}-N-methyl-N-phenyl-benzamide;
N-Butyl-3-{3-[5-(3,5-dichloro-2-hydroxy-phenyl)-[1,3,4]thiadiazol-2-yl]-ureido}-N-phenyl-benzamide;
1-[5-(3,5-Dichloro-2-hydroxy-phenyl)-[1,3,4]thiadiazol-2-yl]-3-[4-(2-methyl-2H-tetrazol-5-yl)-phenyl]-urea;
1-[5-(3,5-Dichloro-2-hydroxy-phenyl)-[1,3,4]thiadiazol-2-yl]-3-[4-(4-dimethylamino-benzoyl)-phenyl]-urea;
1-[5-(3,5-Dichloro-2-hydroxy-phenyl)-[1,3,4]thiadiazol-2-yl]-3-[4-(4-[1,2,3]triazol-2-yl-benzoyl)-phenyl]-urea;
1-[5-(3,5-Dichloro-2-hydroxy-phenyl)-[1,3,4]thiadiazol-2-yl]-3-[4-(4-[1,2,3]triazol-1-yl-benzoyl)-phenyl]-urea;
1-[5-(3,5-Dichloro-2-hydroxy-phenyl)-[1,3,4]thiadiazol-2-yl]-3-[4-(4-[1,2,4]triazol-1-yl-benzoyl)-phenyl]-urea;
1-[5-(3,5-Dichloro-2-hydroxy-phenyl)-[1,3,4]thiadiazol-2-yl]-3-[4-(4-imidazol-1-yl-benzoyl)-phenyl]-urea;
1-[5-(3,5-Dichloro-2-hydroxy-phenyl)-[1,3,4]thiadiazol-2-yl]-3-[4-(pyrrole-1-carbonyl)-phenyl]-urea;
1-[4-(2-Acetyl-pyrrole-1-carbonyl)-phenyl]-3-[5-(3,5-dichloro-2-hydroxy-phenyl)-[1,3,4]thiadiazol-2-yl]-urea;
1-(4-{3-[5-(3,5-Dichloro-2-hydroxy-phenyl)-[1,3,4]thiadiazol-2-yl]-ureido}-benzoyl)-1H-pyrrole-2-carboxylic acid ethyl ester;
4-{3-[5-(2-Hydroxy-5-nitro-phenyl)-[1,3,4]thiadiazol-2-yl]-ureido}-benzoic acid ethyl ester;
4-{3-[5-(2-Hydroxy-4-methoxy-phenyl)-[1,3,4]thiadiazol-2-yl]-ureido}-benzoic acid ethyl ester;
4-{3-[5-(2-Hydroxy-3-methoxy-phenyl)-[1,3,4]thiadiazol-2-yl]-ureido}-benzoic acid ethyl ester;
4-{3-[5-(2-Hydroxy-6-methoxy-phenyl)-[1,3,4]thiadiazol-2-yl]-ureido}-benzoic acid ethyl ester;
Ammonium, 2-{5-[3-(4-ethoxycarbonyl-phenyl)-ureido]-[1,3,4]thiadiazol-2-yl}-4,6-dinitro-phenylate;
4-{3-[5-(5-Chloro-2-hydroxy-3-methoxy-phenyl)-[1,3,4]thiadiazol-2-yl]-ureido}-benzoic acid ethyl ester;
1-(4-Benzoyl-phenyl)-3-[5-(3,5-difluoro-2-hydroxy-phenyl)-[1,3,4]thiadiazol-2-yl]-urea;
4-{3-[5-(3,4,5-Trifluoro-2-hydroxy-phenyl)-[1,3,4]thiadiazol-2-yl]-ureido}-benzoic acid ethyl ester;
1-(4-Benzoyl-phenyl)-3-[5-(2,3,5-trichloro-6-hydroxy-phenyl)-[1,3,4]thiadiazol-2-yl]-urea;
(ii) a pharmaceutically acceptable salt, prodrug, stereoisomer or solvate of (i) thereof.

17. A pharmaceutical composition, comprising a pharmaceutically acceptable carrier and a therapeutically effective amount of a compound of claim 7.

18. A method according to claim 1, wherein the disorder associated with high levels of PAI-1 is a thromboembolic disorder.

19. A method according to claim 18, wherein the thromboembolic disorder is selected from unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

20. A method for treating a thromboembolic disorder, comprising: administering to a patient in need thereof a therapeutically effective amount of a first and second therapeutic agent, wherein the first therapeutic agent is a compound having formula (I) or a pharmaceutically acceptable salt or hydrate thereof and the second therapeutic agent is at least one agent selected from a second PAI-1 inhibitor, a factor Xa inhibitor, an anti-coagulant agent, an anti-platelet agent, a thrombin inhibiting agent, a thrombolytic agent, and a fibrinolytic agent wherein formula (I) is 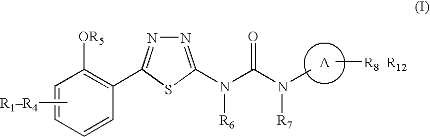
or a pharmaceutically-acceptable salt, prodrug, stereoisomer or solvate thereof, wherein:
A is aryl or heteroaryl;
R₁-R₄ and R₈-R₁₂ are independently selected from hydrogen, halogen, nitro, cyano, alkyl, substituted alkyl, aryl, heteroaryl, cycloalkyl, heterocyclo, —OR₁₃, —SR₁₄, —OC(═O)R₁₃, —C(═O)R₁₃, —CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅;
or any two of R₁-R₄ and R₈-R₁₂ located on neighboring atoms of the ring to which they are attached may be taken together to form a fused ring system in combination with the ring, wherein the fused ring system may be optionally further substituted;
R₅ is hydrogen, alkyl or substituted alkyl;
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and/or R₁₆ is selected independently.

21. A method according to claim 20 wherein the second therapeutic agent is at least one agent selected from warfarin, unfractionated heparin, low molecular weight heparin, synthetic pentasaccharide, hirudin, argatrobanas, aspirin, ibuprofen, naproxen, sulindac, indomethacin, mefenamate, droxicam, diclofenac, sulfinpyrazone, piroxicam, ticlopidine, clopidogrel, tirofiban, eptifibatide, abciximab, melagatran, disulfatohirudin, tissue plasminogen activator, modified tissue plasminogen activator, anistreplase, urokinase, and streptokinase.

22. The method according to claim 21, wherein the second therapeutic agent is at least one anti-platelet agent.

23. The method according to claim 22, wherein the anti-platelet agent is aspirin and clopidogrel.

24. The method according to claim 23, wherein the anti-platelet agent is clopidogrel.

25. An article of manufacture, comprising:
(a) a first container;
(b) a pharmaceutical composition located within the first container, wherein the composition, comprises: a first therapeutic agent, comprising: a compound of any one of formula (I), or a pharmaceutically acceptable salt or hydrate form thereof; and
(c) a package insert stating that the pharmaceutical composition can be used for the treatment of a thromboembolic disorder 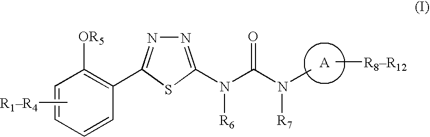
or a pharmaceutically-acceptable salt, prodrug, stereoisomer or solvate thereof, wherein:
A is aryl or heteroaryl;
R₁-R₄ and R₈-R₁₂ are independently selected from hydrogen, halogen, nitro, cyano, alkyl, substituted alkyl, aryl, heteroaryl, cycloalkyl, heterocyclo, —OR₁₃, —SR₁₄, —OC(═O)R₁₃, —C(═O)R₁₃, —CO₂R₁₃, —C(═O)NR₁₄R₁₅, —NR₁₄R₁₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅;
or any two of R₁-R₄ and R₈-R₁₂ located on neighboring atoms of the ring to which they are attached may be taken together to form a fused ring system in combination with the ring, wherein the fused ring system may be optionally further substituted;
R₅ is hydrogen, alkyl or substituted alkyl;
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and/or R₁₆ is selected independently.

26. A article of manufacture according to claim 25, further comprising:
(d) a second container;
wherein components (a) and (b) are located within the second container and component (c) is located within or outside of the second container.

27. A compound of claim 7, or a pharmaceutically acceptable salt or hydrate form thereof, for use in therapy.

28. Use of a compound having formula (I), for the manufacture of a medicament for the treatment of a thromboembolic disorder wherein formula (I) is 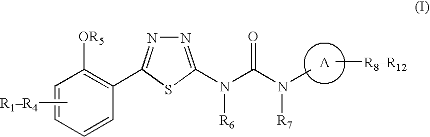
or a pharmaceutically-acceptable salt, prodrug, stereoisomer or solvate thereof, wherein:
A is aryl or heteroaryl;
R₁-R₄ and R₈-R₁₂ are independently selected from hydrogen, halogen, nitro, cyano, alkyl, substituted alkyl, aryl, heteroaryl, cycloalkyl, heterocyclo, —OR₁₃, —SR₁₄, —OC(═O)R₁₃, —C(═O)R₁₃, —CO₂R₁₃, —C(═O)NR₁₄R₁₅, —NR₁₄R₅, —S(═O)R₁₃, —SO₂R₁₃, —SO₂NR₁₄R₁₅, —NR₁₆SO₂NR₁₄R₁₅, —NR₁₆SO₂R₁₃, —NR₁₆C(═O)R₁₃, —NR₁₆CO₂R₁₃, and —NR₁₆C(═O)NR₁₄R₁₅;
or any two of R₁-R₄ and R₈-R₁₂ located on neighboring atoms of the ring to which they are attached may be taken together to form a fused ring system in combination with the ring, wherein the fused ring system may be optionally further substituted;
R₅ is hydrogen, alkyl or substituted alkyl;
R₆ and R₇ are independently hydrogen or C₁₋₆alkyl; and
R₁₃, R₁₄, R₁₅ and R₁₆ are independently selected from hydrogen, alkyl, substituted alkyl, aryl, heteroaryl, cycloalkyl and heterocyclo, wherein each instance of R₁₃, R₁₄, R₁₅ and/or R₁₆ is selected independently.
